(19) United States
(12) Patent Application Publication
(10) Pub. No.: US 2011/0191916 A1
(43) Pub. Date: Aug. 4, 2011

(54) COMPOSITIONS FOR PRODUCTION OF SOYBEAN WITH ELEVATED OIL CONTENT

(76) Inventors: Mark A. Erickson, Slater, IA (US); Thomas Horejsi, Madison, WI (US); Joseph R. Byrum, West Des Moines, IA (US); Donghong Pei, Northfield, MN (US)

(21) Appl. No.: 12/600,231

(22) PCT Filed: May 29, 2008
(86) PCT No.: PCT/US08/65123

§ 371 Date: Feb. 23, 2011

Related U.S. Application Data
(60) Provisional application No. 60/932,433, filed on May 31, 2007.

(51) Int. Cl. A01H 5/10; C10L 1/00; A23D 9/00; A23L 1/20; A23J 1/00; C12Q 1/02; C10M 101/04; C07K 14/415; C11B 1/00; A01H 5/00; A23K 1/14

(52) U.S. Cl. 800/300; 44/307; 426/601; 426/634; 426/656; 435/29; 508/216; 530/378; 554/9; 800/301; 800/302; 800/312

(57) ABSTRACT

The present invention is in the field of plant breeding and genetics, as it pertains to the soybean plant, Glycine max L. More specifically, the invention relates to soybean plants capable of producing seed with total oil level in excess of 23% wherein the plant comprises one or more transgenic trait, as well as to non-transgenic or transgenic soybean plants capable of producing seed with total oil level in excess of 26%. Plant parts including seeds are also provided, as well as methods for producing food, feed, fuel, industrial products, protein products, and oil products. Methods of detection of high oil seeds are also provided.

### CROSS-REFERENCE TO RELATED APPLICATIONS

This application claims the benefit under 35 U.S.C. §119(e) of U.S. Provisional Application No. 60/932,433 filed May 31, 2007. The entirety of the application is hereby incorporated by reference.

### BACKGROUND OF THE INVENTION

1. Field of Invention

The present invention is in the field of soybean breeding. Specifically, the present invention relates to soybean plants capable of producing a soybean seed with total oil level in excess of 23% by weight and said plant with one or more transgenic trait. In addition the invention relates to non-transgenic or transgenic soybean plants with total oil level in excess of 26%.

2. Background of Invention

Seventy-five percent of all edible oil consumed in the United States is soybean oil with over 14 billion pounds of soybean oil produced annually. Soybean oil accounts for the largest percentage of edible oil consumed worldwide and is used in a broad range of food manufacturing applications including the production of liquid shortening, margarines, soft spreads and low-fat spreads. It is an important ingredient in products such as salad dressings, non-dairy creamers, whipped toppings, breakfast cereals, ice cream, soups, confectionery products, cooking oils, frozen dairy desserts, peanut butter, sandwich spreads and snack foods. In addition, soybean oil is used for industrial purposes with over 600 million pounds of the soybean oil produced for non-edible applications such as the production of industrial materials, including fatty acids, soaps, inks, paints, varnishes, resins, plastics, and fuel.

Soybean seed oil levels are highly impacted by environment. Oil concentration increases with decreasing latitude, therefore, soybeans in early maturity group soybeans (00-I) generally have lower oil levels than later maturing soybeans (Yaklich et al. 2002). The decrease in oil concentrations is attributed to lower temperatures and shorter growing season (Piper and Boote 1999). In addition, soybeans cultivated under drought stress tend to produce seeds with decreased protein and increased oil (Specht et al. 2001).

Six introductions, ‘Mandarin,’ ‘Manchu,’ ‘Mandarin (Ottawa)’, ‘Richland,’ ‘AK’ (Harrow), and ‘Mukden,’ contributed nearly 70% of the germplasm represented in 136 cultivar releases. Soybean breeders utilize both exotic germplasm and known varieties with high oil to increase oil levels in breeding programs. Breeding with exotic species widens the genetic base and contributes novel sources of oil genes, but progeny is generally not well suited for agronomic conditions and require significant backcrossing to recover a desirable plant type. In addition, oil levels can be highly variable for progeny developed from crossing adapted and exotic soybean plants, making it difficult to evaluate gains in seed oil levels (Scott and Kephart 1997).

Elite soybean plants comprising increased oil in the seed and transgenic traits, such as herbicide resistance, provides a useful soybean product that is currently not available to farmers and consumers. The present invention provides methods and compositions for the discovery and breeding of soybean plants capable of producing seed with elevated levels of oil, wherein oil levels are in excess of 23% and said plant comprises one or more transgenic traits, as well as non-transgenic or transgenic soybean lines that produce seeds comprising at least 26% oil.

### SUMMARY OF INVENTION

The present invention provides a soybean plant capable of producing seed with oil content in excess of 23% and said plant comprising one or more transgenic traits. Also provided are the parts of said plant, including, but not limited to, pollen, an ovule, a cell and a seed. Further provided is a tissue culture or regenerable cells of the plant, wherein the tissue culture regenerates soybean plants capable of expressing all the physiological and morphological characteristics of the plant.

In another aspect, the invention provides a soybean plant of the invention comprising a transgene. The transgene may in one embodiment be defined as conferring at least one preferred property to the soybean plant selected from the group consisting of herbicide tolerance, increased yield, insect control, fungal disease resistance, virus resistance, nematode resistance, bacterial disease resistance, mycoplasma disease resistance, modified fatty acid composition, increased oil production, modified amino acid composition, modified protein production, increased protein production, increased carbohydrate production, germination and seedling growth control, enhanced animal and human nutrition, low raffinose, drought and/or environmental stress tolerance, altered morphological characteristics, increased digestibility, industrial enzymes, pharmaceutical proteins, peptides and small molecules, improved processing traits, improved flavor, nitrogen fixation, hybrid seed production, reduced allergenicity, biopolymers, biofuels, or any combination of these. The expression of a transgene of agronomic interest is desirable in order to confer an agronomically important trait. A gene of agronomic interest (a transcribable polynucleotide molecule) that provides a beneficial agronomic trait to crop plants may be, for example, including, but not limited to genetic elements comprising herbicide resistance (U.S. Pat. Nos. 6,803,501; 6,448,476; 6,248,876; 6,225,114; 6,107,549; 5,866,775; 5,804,425; 5,633,435; 5,463,175), increased yield (U.S. Pat. Nos. RE 38,446; 6,716,474; 6,663,906; 6,476,295; 6,441,277; 6,423,828; 6,399,330; 6,372,211; 6,235,971; 6,222,098; 5,716,837), insect control (U.S. Pat. Nos. 6,809,078; 6,713,063; 6,686,452; 6,657,046; 6,645,497; 6,642,030; 6,639,054; 6,620,988; 6,593,293; 6,555,655; 6,538,109; 6,537,756; 6,521,442; 6,501,009; 6,468,523; 6,326,351; 6,313,378; 6,284,949; 6,281,016; 6,248,536; 6,242,241; 6,221,649; 6,177,615; 6,156,573; 6,153,814; 6,110,464; 6,093,695; 6,063,756; 6,063,597; 6,023,013; 5,959,091; 5,942,664; 5,942,658, 5,880,275; 5,763,245; 5,763,241), fungal disease resistance (U.S. Pat. Nos. 6,653,280; 6,573,361; 6,506,962; 6,316,407; 6,215,048; 5,516,671; 5,773,696; 6,121,436; 6,316,407; 6,506,962), virus resistance (U.S. Pat. Nos. 6,617,496; 6,608,241; 6,015,940; 6,013,864; 5,850,023; 5,304,730), nematode resistance (U.S. Pat. No. 6,228,992), bacterial disease resistance (U.S. Pat. No. 5,516,671), plant growth and development (U.S. Pat. Nos. 6,723,897; 6,518,488), starch production (U.S. Pat. Nos. 6,538,181; 6,538,179; 6,538,178; 5,750,876; 6,476,295), modified oils production (U.S. Pat. Nos. 6,444,876; 6,426,447; 6,380,462), high oil production (U.S. Pat. Nos. 6,495,739; 5,608,149; 6,483,008; 6,476,295), modified fatty acid content (U.S. Pat. Nos. 6,828,475; 6,822,141; 6,770,465; 6,706,950; 6,660,849; 6,596,538; 6,589,767; 6,537,750; 6,489,461; 6,459,018), high protein production (U.S. Pat. No. 6,380,466), fruit ripening (U.S. Pat. No. 5,512,466), enhanced animal and human nutrition (U.S. Pat. Nos. 6,723,837; 6,653,530; 6,5412,59; 5,985,605; 6,171,640), biopolymers (U.S. Pat. No. RE 37,543; 6,228,623; 5,958,745 and U.S. Patent Publication No. US20030028917), environmental stress resistance (U.S. Pat. No. 6,072,103), pharmaceutical peptides and secretable peptides (U.S. Pat. Nos. 6,812,379; 6,774,283; 6,140,075; 6,080,560), improved processing traits (U.S. Pat. No. 6,476,295), improved digestibility (U.S. Pat. No. 6,531,648) low raffinose (U.S. Pat. No. 6,166,292), industrial enzyme production (U.S. Pat. No. 5,543,576), improved flavor (U.S. Pat. No. 6,011,199), nitrogen fixation (U.S. Pat. No. 5,229,114), hybrid seed production (U.S. Pat. No. 5,689,041), fiber production (U.S. Pat. Nos. 6,576,818; 6,271,443; 5,981,834; 5,869,720) and biofuel production (U.S. Pat. No. 5,998,700). The genetic elements, methods, and transgenes described in the patents listed above are incorporated herein by reference.

In still another aspect, the invention provides a soybean plant comprising a specialty trait. The specialty trait may in one embodiment be defined as conferring preferred property to the soybean plant selected from the group consisting of less than 4% linolenic acid, less than 11% palmitic acid, greater than 14% stearic acid, greater than 20% oleic acid, less than 35% linoleic, greater than 6% gamma linolenic acid, greater than 8% docosahexaenoic acid, greater than 8% eicosapentaenoic acid, greater than 8% docosapentaenoic acid, 2% stearidonic acid or any combinations of these.

Another aspect of the invention is a method of producing an industrial products comprising: (a) obtaining a soybean seed of the invention, (b) planting and growing said seed into a mature plant, (c) harvesting seed from said plant, and (d) preparing an industrial product from said harvested seed. In certain embodiments of the invention, the industrial products may comprise fuels, lubricants, resins, binders, glues, adhesives, inks, paints, fungicides, disinfectants, rubber, cosmetics, caulking compounds, wallboard, anti-foam agents, alcohol, waxes, solvents, or films.

Another aspect of the invention is a method of producing a food or feed product comprising: (a) obtaining a soybean seed of the invention, (b) planting and growing said seed into a mature plant, (c) harvesting seed from said plant, and (d) preparing a food or feed product from said harvested seed. In certain embodiments of the invention, the food or feed products may comprise animal feed, pharmaceuticals, soy milk, tofu, roasted soybeans, baby foods, soynut butter, or other soy derivatives.

Yet another aspect of the invention is a method of producing an oil product comprising: (a) obtaining a soybean seed of the invention, (b) planting and growing said seed into a mature plant, (c) harvesting seed from said plant, and (d) preparing an oil product from said harvested seed. In certain embodiments of the invention, the oil product may comprise food oil, feed, fuel, resins, disinfectants, fungicides, rubber, fuel, paint, cosmetics, pharmaceuticals, inks and lubricants. Examples of food oils are cooking oil, emulsified products (e.g. mayonnaise, shortening, margarine, and salad dressing), and intermediate moisture foods (e.g. dog foods).

Still yet another aspect of the invention is a method of producing a protein product comprising: (a) obtaining a soybean seed of the invention, (b) planting and growing said seed into a mature plant, (c) harvesting seed from said plant, and (d) preparing a protein product from said harvested seed. In certain embodiments of the invention, the protein product may comprise protein isolate, meal, flour, soybean hulls for food or feed.

Another aspect of the invention is a method for detecting the presence of a high oil soybean seed in a population of seed, comprising: (a) obtaining a population of soybean seed; and (b) detecting in said population the presence of a seed wherein the total oil content of the seed is between 25-33%. In certain embodiments the detection method comprises one or more of: Near Infrared Reflectance (NIR), Near-Infrared Transmittance (NIT), Nuclear Magnetic Resonance (NMR), and solvent extraction.

### BRIEF DESCRIPTION OF THE FIGURES

FIG. 1. A population from a cross between high oil soybean lines derived from mutagenesis.

### DETAILED DESCRIPTION OF THE INVENTION

The invention overcomes the deficiencies of the prior art by providing soybean varieties that produce seeds comprising at least 23% oil by weight, wherein the plants and seeds comprise one or more transgenic traits. Soybean plants that produce seeds comprising at least 26% oil are also provided. Additionally, the parts of said plant, including, but not limited to, pollen, an ovule, a cell and a seed, are provided. Further provided is a tissue culture or regenerable cells of the plant, wherein the tissue culture regenerates soybean plants capable of expressing all the physiological and morphological characteristics of the plant. The prior art has failed to provide plants of such a variety. By describing the production of such plants and providing these plants, the invention now allows the preparation of a potentially unlimited number of novel soybean varieties exhibiting such a described high oil trait, optionally in conjunction with one or more transgenic traits. This is because, once parent plants for the production of the variety are identified, then the described oil attribute as well as one or more transgenic traits can be transferred to other varieties with appropriate backcross and selection to maintain the desirable traits, as is described herein below.

There are numerous steps in the development of any novel, desirable plant germplasm, such as the lines described herein or varieties derived therefrom using the methods of the invention. Plant breeding begins with the analysis and definition of problems and weaknesses of the current germplasm, the establishment of program goals, and the definition of specific breeding objectives. The next step is selection of germplasm that possess the traits to meet the program goals. The goal is to combine in a single variety an improved combination of desirable traits from the parental germplasm. In addition to a high oil, these important traits may include, for example, resistance to diseases and insects, better stems and roots, tolerance to drought and heat, better agronomic quality, resistance to herbicides, and improvements in various compositional traits.

The choice of breeding or selection methods depends on the mode of plant reproduction, the heritability of the trait(s) being improved, and the type of variety used commercially (e.g., F₁ hybrid variety, pureline variety, etc.). For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, recurrent selection and backcrossing. Methods that may be employed in connection with the instant invention are described in detail herein below.

### I. Plants of the Invention

This invention provides soybean plants with increased oil levels in their seed, including soybean plants with seed oil levels greater than 23%, including about 23-35% or about 25-33% seed oil, and wherein the plant comprises one or more transgenic traits. In addition, the invention provides soybean plants with seed oil levels greater than 26%, including between about 26-35% or about 28-33% seed oil. Seed of such plants, and seed of a subsequent generation of such plants, are also provided.

One aspect of the current invention is therefore directed to the plants and parts thereof and method for using these plants and plant parts. Plant parts include, but are not limited to, pollen, an ovule and a cell. The invention further provides tissue cultures of regenerable cells of these plants, which cultures regenerate soybean plants capable of expressing all the physiological and morphological characteristics of the starting variety. Such regenerable cells may include embryos, meristematic cells, pollen, leaves, roots, root tips or flowers, or protoplasts or callus derived therefrom. Also provided by the invention are soybean plants regenerated from such a tissue culture, wherein the plants are capable of expressing all the physiological and morphological characteristics of the starting plant variety from which the regenerable cells were obtained.

II. Production of Soybean Varieties with Elevated Oil Content

The present invention also provides methods to produce soybean plants with elevated oil content in seed. For instance, one method involves inducing mutations to confer elevated oil levels in seed. Another method, for instance, involves crossing two soybean parents with high seed oil levels to further increase oil content of seed. The high oil parents in the second approach may for instance be germplasm-screened for oil level; soybeans expressing transgenes to increase oil levels in seed; or may be derived from mutagenesis as described, among other sources.

As indicated, one method, mutagenesis, may involve multiple cycles of gamma radiation, effective at increasing oil level of seed. A stepwise oil increase may be detected from each cycle of radiation. For instance, oil content increased as much as 3.8% compared to the mother line from the first cycle of radiation. Oil content may be further elevated from a second cycle of radiation, for instance to as much as 29.2%.

Another method to obtain soybean plants producing seeds comprising the described oil content may involve crosses between selected high oil parents to additionally elevate seed oil levels. Such crosses combine differing high oil genes from different soybean sources. Subsequent self-pollinating of progeny allows for lines comprising multiple parental high oil genes or traits, potentially producing progeny with even higher oil levels. The parents for the cross may for instance be elite high oil lines, mutants developed from the first approach, or soybeans expressing transgenes to increase oil levels in seed, among other sources. Examples of high oil elite lines that are commercially available to growers or soybean breeders include, for instance, Asgrow® Brand Soybeans: AG2107, DKB31-51, DKB22-52, AG2403, DKB25-51, DKB26-53, DKB28-52, AG3005, AG4403, DKB44-51, AG4801, and AG5903.

Following the crossing high oil parents, further breeding steps may comprise,

among others: (a) crossing a first high oil parent to a second high oil parent, (b) screening the progeny for high oil using NIT (Near-Infrared Transmittance) or other method, and (c) selecting one or more progeny plant containing the desired trait of high oil levels in the seed. High oil seeds may be detected. Selected high oil lines may be used in the further breeding efforts to develop new lines with high oil seed.

### III. Utilization of Soybean Plants

Generally, the following steps are used to process soybean seed: preparation, cracking and dehulling, conditioning, milling, flaking or pressing, extracting, degumming, refining, bleaching, and deodorizing. Each of these steps is discussed in more detail herein below. The discussion details the process for each of the steps used currently in an exemplary commercial application. A person of ordinary skill in the art would know that such steps could be combined, used in a different order or otherwise modified.

Generally, the preparation step includes the initial cleaning process, which removes tones, dirt, sticks, worms, insects, metal fragments, and other debris collected during the harvest and storage of the seeds. Extraneous matter as described above can affect the quality of the final seed oil by containing compounds that negatively impact its chemical stability, Preferably, ripe, unbroken seeds having reduced levels of chlorophyll and reduced levels of free fatty acids are used.

After the preparation step, the seeds are cracked and dehulled. Cracking and dehulling can be accomplished in a variety of ways, which are well known in the art. For example, the seeds can be cracked and dehulled using a seed cracker, which mechanically breaks the seeds and releases hulls and directly exposes the inner seed meat to air. After cracking, the hulls can be separated from the seed meats by a dehuller. In one aspect, the dehuller can separate the hulls from the seed meats due to the density difference between the hulls and the seeds; the hulls are less dense than the seed meats. For examples, aspiration will separate the hulls from the cracked seed meats. Dehulling reduces the crude fiber content, while increasing the protein concentration of the extracted seed meats. Optionally, after dehulling, the hulls can be sieved to recover the fines generated in the cracking of the seeds. After recovery, the fines can be added back to the seed meats prior to conditioning.

Once the seeds are cracked, the oxygen exposure of the seed meats can optionally be minimized, which would reduce oil oxidation and improve oil quality. Furthermore, it will be understood by persons skilled in the art that minimization of oxygen exposure may occur independently at each of the subsequently disclosed oilseed processing steps.

Once the seeds are cracked and dehulled, they are conditioned to make the seed meats pliable prior to further processing. Furthermore, the conditioning ruptures oil bodies. Further processing, in terms of flaking, grinding or other milling technology is made easier by having pliable meats at this stage. Generally, the seed meats have moisture removed or added in order to reach a 6-10 wt. % moisture level. If moisture is removed, this process is called toasting and if moisture is added, this process is called cooking. Typically, the seed meats are heated to 40-90° C. with steam which is dry or wet depending on the direction of adjustment of moisture content of the seed meats. In some instances, the conditioning step occurs under conditions minimizing oxygen exposure or at lower temperature for seeds having high poly unsaturated fatty acid (PUFA) levels.

Once the seed meats are conditioned, they can be milled to a desired particle size or flaked to a desired surface area. In certain cases, the flaking or milling occurs under conditions minimizing oxygen exposure. Flaking or milling is done to increase the surface area of the seed meats and also to rupture the oil bodies thereby facilitating a more efficient extraction. Many milling technologies are appropriate and are well known in the art. The considerations when choosing a method of milling and a particle size for the ground seed are contingent upon, but not limited to, the oil content in the seed and the desired efficiency of the extraction of the seed meats or the seed. When flaking the seed meats, the flakes are typically from about 0.1 to about 0.5 mm thick; from about 0.1 to about 0.35 mm thick; from about 0.3 to 0.5 mm thick; or from about 0.2 to about 0.4 mm thick.

Optionally, after the seed meats are milled, they can be pressed. Typically, the seed meats are pressed when the oil content of the seed meats is greater than about 30 wt. % of the seeds. However, seeds with higher or lower oil contents can be pressed. The seed meats can be pressed, for example, in a hydraulic press or mechanical screw. Typically, the seed meats are heated to less than about 55° C. upon the input of work. When pressed, the oil in the seed meats is pressed through a screen, collected and filtered. The oil collected is the first press oil. The seed meats from after pressing are called seed cake; the seed cake contains oil and can be subjected to solvent extraction (e.g. Sallee, 1968). The soy meal is the product of solvent extraction and is often used as a protein source for animal feed.

After milling, flaking or optional pressing, the oil can be extracted from the seed meats or seed cake by contacting them with a solvent. Preferably, n-hexane or iso-hexane is used as the solvent in the extraction process. Typically, the solvent is degassed prior to contact with the oil. This extraction can be carried out in a variety of ways, which are well known in the art. For example, the extraction can be a batch or continuous process and desirably is a continuous counter-current process. In a continuous counter-current process, the solvent contact with the seed meat leaches the oil into the solvent, providing increasingly more concentrated miscella (i.e., solvent-oil), while the marc (i.e., solvent-solids) is contacted with the miscella of decreasing concentration. After extraction, the solvent is removed from the miscella in a manner well known in the art. For example, distillation, rotary evaporation or a rising film evaporator and steam stripper can be used for removing the solvent. After solvent removal, if the crude oil still contains residual solvent, it can be heated at about 95° C. and about 60 mm Hg.

The above processed crude oil contains hydratable and non-hydratable phosphatides. Accordingly, the crude oil is degummed to remove the hydratable phosphatides by adding water and heating to from about 40° to about 75° C. for approximately 5-60 minutes depending on the phosphatide concentration. Optionally, phosphoric acid and/or citric acid can be added to convert the non-hydratable phosphatides to hydratable phosphatides. Phosphoric acid and citric acid form metal complexes, which decreases the concentration of metal ions bound to phosphatides (metal complexed phosphatides are non-hydratable) and thus, convert non-hydratable phosphatides to hydratable phosphatides. Generally, if the phosphoric acid and/or citric acid are added in the degumming step about 1 to about 5 wt. %; preferably, about 1 wt. % or about 2 wt. %; more preferably, about 1.5 to about 2 wt. % are used. This process is optionally carried out by degassing the water and phosphoric acid before contacting them with the oil.

Furthermore, the crude oil contains free fatty acids (FFAs), which can be removed by a chemical (e.g., caustic) refining step. When FFAs react with basic substances (e.g., caustic) they form soaps that can be extracted in aqueous solution. Thus, the crude oil is heated to about 40 to about 75° C. and NaOH is added with stiffing and allowed to react for approximately 10 to 45 minutes. This is followed by stopping the stiffing while continuing heat, removing the aqueous layer, and treating the neutralized oil to remove soaps. The oil is treated by water washing the oil until the aqueous layer is of neutral pH, or by treating the neutralized oil with a silica or ion exchange material. The oil is dried at about 95° C. and about 10 mmHg In some instances, the caustic solution is degassed before it contacts the oil.

Alternatively, rather than removing FFAs from the oil by chemical refining, the FFAs may by removed by physical refining. For example, the oil can be physically refined during deodorization. When physical refining is performed, the FFAs are removed from the oil by vacuum distillation performed at low pressure and relatively higher temperature. Generally, FFAs have lower molecular weights than triglycerides and thus, FFAs have lower boiling points and can be separated from triglycerides based on this boiling point difference and through the aid of nitrogen or steam stripping used as an azeotrope or carrier gas to sweep volatiles from the deodorizers.

Typically, when physical refining rather than chemical refining is performed, oil processing conditions are modified to achieve similar final product specifications. For example, when an aqueous acidic solution is used in the degumming step, a higher concentration of acid (e.g., up to about 100% greater concentration, preferably about 50 to 100% greater concentration) may be needed due to the greater concentration of non-hydratable phosphatides that could otherwise be removed in a chemical refining step. In addition, a greater amount of bleaching material (e.g., up to about 100% or greater amount, preferably about 50 to about 100% greater amount) is used.

Before bleaching, citric acid (50 wt. % solution) can be added at a concentration of about 0.01 to about 5 wt. % to the degummed oil and/or chemically refined oil. This mixture can then be heated at a temperature of about 35° to about 65° C. and a pressure of about 1 to about 760 mm Hg for about 5 to about 60 minutes.

The degummed oil and/or chemically refined oil is subjected to an absorption process (e.g., bleached) to remove peroxides, oxidation products, phosphatides, keratinoids, chlorophylloids, color bodies, metals, and remaining soaps formed in the caustic refining step or other processing steps. The bleaching process comprises heating the degummed oil or chemically refined oil under vacuum of about 0.1 mmHg to about 200 mm Hg and adding a bleaching material appropriate to remove the above referenced species (e.g., neutral earth (commonly termed natural clay or fuller's earth), acid-activated earth, activated clays and silicates) and a filter aid, whereupon the mixture is heated to about 75° to 125° C., and the bleaching material is contacted with the degummed oil and/or chemically refined oil for about 5 to 50 minutes. It can be advantageous to degas the bleaching material before it contacts the refined oil. The amount of bleaching material used is from about 0.25 to about 3 wt. %, preferably about 0.25 to about 1.5 wt. %. After heating, the bleached oil or refined, bleached oil is filtered and deodorized.

The bleached oil or refined, bleached oil is deodorized to remove compounds with strong odors and flavors as well as remaining FFAs. The color of the oil can be further reduced by heat bleaching at elevated temperatures. Deodorization can be performed by a variety of techniques including batch and continuous deodorization units such as batch stir tank reactors, falling film evaporators, wiped film evaporators, packed column deodorizers, tray type deodorizers, and loop reactors. Typically, a continuous deodorization process is preferred. Generally, deodorization conditions are performed at about 160° to 270° C. and about 0.002 to about 1.4 kPa. For a continuous process, particularly in a continuous deodorizer having successive trays for the oil to traverse, a residence time of up to 2 hours at a temperature from about 170° to about 265° C.; a residence time of up to about 30 minutes at a temperature from about 240° to about 250° C. is preferred. Deodorization conditions ca use carrier gases for the removal of volatile compounds (e.g., steam, nitrogen, argon, or any other gas that does not decrease the stability or the quality of the oil).

Furthermore, when physical rather than chemical refining is used, a greater amount of FFAs are removed during the deodorization step, and the deodorizer conditions are modified to facilitate the removal of FFAs. For examples, the temperature is increased by about 25° C.; oils can be deodorized at temperature ranging from about 165° to about 300° C. In particular, oils can be deodorized at temperatures ranging from about 250° to about 280° C. or about 175° to about 205° C. In addition, the retention time of the oil in the deodorizer is increased by up to about 100%. For example, the retention time can range from less than about 1, 5, 10, 30, 60, 90, 100, 110, 120, 130, 150, 180, 210, or 240 minutes. Additionally, the deodorizer pressure can be reduced to less than about 3×10⁻⁴, 1×10⁻³, 5×10⁻³, 0.01, 0.02, 0.03, 0.04, 0.05, 0.06, 0.07, 0.08, 0.09, or 0.1 kPa. The deodorization step results in a refined, bleached, and deodorized (RBD) oil.

Optionally, RBD oils can be stabilized by partial hydrogenation and/or by the addition of stabilizers or by minimizing the removal or degradation of microcomponents that aid in the maintaining of oil stability and quality. Partial hydrogenation stabilizes oil by reducing the number of double bonds in the fatty acids contained in the oil and thus reducing the chemical reactivity of the oil. Stabilizers generally act to intercept free radicals formed during oxidation. Notably, partial hydrogenation can increase the concentration of undesirable trans-fatty acids and the present invention provides a soybean with oil content precluding the need for a hydrogenation step.

Alternatively, oil extraction can be performed on a laboratory scale, where individual seeds or seeds of individual plants are analyzed. All exemplary operations may be performed in an inert atmosphere (under an active purge with nitrogen) utilizing a glove bag, a glove box or airless transfer schlenk line techniques. Thus, whole seeds may be placed in mega-grinder capsules under inert conditions and sealed with an airtight cap. The sealed capsules are then removed from the inert atmosphere and milled/ground on the mega-grinder platform. The capsules are then returned to an inert atmosphere where they can be opened and further processing is initiated. All solvents and solutions may be previously degassed with a subsurface sparging of nitrogen. All vessels that are brought into an inert environment chamber may be degassed so adequate purging of the container is possible.

For the milling procedure, the glove bag is purged, for instance three times with nitrogen, and about 20 g of seed are weighed out and added to TEFLON capsules for the mega-grinder. Capsules are filled so the total weight of seed was approximately 200 grams. O-ring seals are placed on the capsules and tape is applied to the lids of the containers for added protection from the diffusion of air into the capsules. Sealed capsules are stored at about 4° C. for two hours prior to milling. Seeds are milled at about 1100 RPM for 45 seconds.

Alternatively, for the cracking, dehulling, and milling procedure, seeds may be cracked, for instance twice, in the cracker (in the inert atmosphere). The cracked seed and hulls are passed from a series of sieves to separate the fines. The seeds and hulls are then aspirated to remove the hulls. About 20 g of dehulled seeds were added to TEFLON capsules for the mega-grinder. Capsules are filled so the total weight of seed was approximately 200 grams. O-ring seals are placed on the capsules and tape is applied to the lids of the containers for added protection from the diffusion of air into the capsules. Sealed capsules may be stored at about 4° C. for two hours prior to milling. Seeds may be milled at about 1100 RPM for 45 seconds.

The capsules may then be placed in a glove bag, and purged, for instance three times, with nitrogen. The capsules may then be opened. A glass thimble for the soxhlet extractor is filled with the ground seed, the soxhlet extractor is removed from the glove box, about 750 ml of hexane is added to a round bottom flask and the ground seed may be extracted for about 7 hours. The miscella is then transferred to a short path distillation apparatus and a vacuum distillation may be performed to remove the hexane to yield the crude oil.

The crude oil may then be charged into a jacketed reactor and heated, for instance to about 50°±3° C. The crude oil is stirred with a magnetic stir bar at about 350 RPM. Once the oil temperature is at about 50° C., an about 5% citric acid solution is added at about 2% (on wt/wt oil basis) and the mixture is heated at about 50°±3° C. for about 30 minutes. The temperature is then increased to about 67°±3° C. When this temperature is reached, the contents are removed and centrifuged. The oil phase is removed and placed back into the jacketed reactor. The reactor is heated to about 62°±3° C. A 5% phosphoric acid solution is added at about 2.0% (based on wt/wt oil basis). The mixture is stirred at about 350 RPM for about 30 minutes. The total acid content is determined and about 1.10 equivalents (based on total acid measurement) of an about 11 wt % NaOH solution is added. The contents of the reactor are maintained at about 62°±3° C. and stirred for about 15 minutes at about 350 RPM. The temperature is raised to about 73±3° C. Once this temperature is reached, the mixture is removed and centrifuged.

For water washing, the oil is returned to the reactor and heated to about 73±3° C. and stirred at about 350 RPM with about 15% HPLC grade water (wt/wt basis) for about 10 minutes. The contents of the reactor are removed and centrifuged.

For bleaching, the oil is transferred into the reactor and heated at about 60°±3° C. and about 2% (wt/wt basis) of an about 5% citric acid solution is added and stirred at about 350 RPM for about 15 minutes. The, about 0.2-0.2 wt % Trisyl® S615 (W.R. Grace, Baltimore, Md., USA) is added and stirred for about 15 minutes. Then, about 0.75-1.25 wt % of Tonsil Grade 105 bleaching clay is added and the pressure in the reactor is reduced to 25 mmHg. The content are heated to about 110±2° C. and stirred at about 350 RPM for about 30 minutes. The mixture is cooled to about 72±3° C. and is filtered in a separate vessel.

For deodorization, the filter oil is placed in a round bottom flask equipped with a claisen head that contains a subsurface gas bleed tube and a vacuum port adaptor. The nitrogen flow is initiated and the vacuum is maintained below 100 millitorr for about 30 minutes at about 255±5° C. Alternatively, the nitrogen flow is initiated and the vacuum is maintained below 100 millitorr for about 2 hours at about 220±5° C. The oil is then cooled to room temperature with an active nitrogen purge.

The soybean meal is a bi-product from the solvent extraction process for oil. The different types of soybean meals are characterized mainly by their protein content and the extent of heat treatment applied in their production to inactivate anti-nutritional factors. If the soybeans are extracted without dehulling, or if the hulls are added back after extraction, the meal will contain about 44% protein. Meals produced from dehulled beans contain approximately 50% protein. The extent of heat treatment or toasting is measured in terms of residual urease activity or as the solubility of the protein under specified conditions. The optimal degree of toasting depends on the final application. Thus, meal for poultry rations must be toasted much more thoroughly than meal for use in cattle feeds.

Protein products intended for human consumption, such as flour, are defatted. Defatting meal is essentially soybean meal which has been ground to the appropriate mesh size. The starting material is dehulled beans and strict sanitary requirements are applied to processing, storage and packaging conditions, in order to secure the microbiological quality of the final product (e.g. total microbial count). In addition, a large variety of products, differing in their lipid content are produced by adding-back soybean oil and/or lecithin to defatted flour or grits at specified levels (refatting). Products containing about 70% protein are prepared from defatted meal by selective extraction of the soluble carbohydrates (sugars). Extraction with aqueous alcohol is the most common process, but other methods of production are available. The concentrates are essentially bland.

Even higher concentrations of protein, in the order of 96%, are obtained by selective solubilization of the protein (e.g. alkaline extraction), followed by purification of the extract and precipitation of the protein (e.g. by acidification to the isoelectric point). Isoelectric isolates are insoluble in water and have practically no functional features. They can be converted to sodium, potassium or calcium proteinates by dissolving isoelectric protein in the appropriate base and spray-drying the solution. Sodium and potassium proteinates are water soluble. They are used mainly for their functional properties, such as emulsification or foaming. One of the by-products of the protein isolation process, the insoluble residue, is also commercialized for its remarkable water absorption capacity and as a source of dietary fiber.

Generally, the following steps are used to process soy feed: combining soy flour, sugar and liquid to provide a mixture; gelatinizing the carbohydrate in the soy flour that is present in the mixture; then reacting the yeast with the mixture, preferably at a temperature of from about 15 to about degree 50° C., and terminating the chemical reactions. The discussion details the process for each of the steps used currently in an exemplary commercial application. A person of ordinary skill in the art would know that the steps could be combined, used in a different order or otherwise modified.

Soybeans have many industrial uses. One common industrial usage for soybeans is the preparation of binders that can be used to manufacture composites. For example, wood composites may be produced using modified soy protein, a mixture of hydrolyzed soy protein and PF resins, soy flour containing powder resins, and soy protein containing foamed glues. Soy-based binders have been used to manufacture common wood products such as plywood for over 70 years. Although the introduction of urea-formaldehyde and phenol-formaldehyde resins has decreased the usage of soy-based adhesives in wood products, environmental concerns and consumer preferences for adhesives made from a renewable feedstock have caused a resurgence of interest in developing new soy-based products for the wood composite industry.

Preparation of adhesives represents another common industrial usage for soybeans. Examples of soy adhesives include soy hydrolyzate adhesives and soy flour adhesives. Soy hydrolyzate is a colorless, aqueous solution made by reacting soy protein isolate in a 5% sodium hydroxide solution under heat (120° C.) and pressure (30 psi). The resulting degraded soy protein solution is basic (pH 11) and flowable (approximately 500 cps) at room temperature. Soy flour is a finely ground, defatted meal made from soybeans. Various adhesive formulations can be made from soy flour, with the first step commonly requiring dissolving the flour in a sodium hydroxide solution. The strength and other properties of the resulting formulation will vary depending on the additives in the formulation. Soy flour adhesives may also potentially be combined with other commercially available resins.

Soybean oil may find application in a number of other industrial uses. Soybean oil is the most readily available and one of the lowest-cost vegetable oils in the world. Common industrial uses for soybean oil include use as components of anti-static agents, caulking compounds, disinfectants, fungicides, inks, paints, protective coatings, wallboard, anti-foam agents, alcohol, margarine, paint, ink, rubber, shortening, fuel, cosmetics, etc. Soybean oils have also for many years been a major ingredient in alkyd resins, which are dissolved in carrier solvents to make oil-based paints. The basic chemistry for converting vegetable oils into an alkyd resin under heat and pressure is well understood to those of skill in the art.

Soybean plants that produce soybean seeds with elevated oil levels may be of particular use for production of biofuel and lubricants. Biofuel may be any fuel that is derived from biomass, for instance comprising at least 50% by volume of material such as soybean oil. Increased oil content can allow production of fuel or lubricant with enhanced utility, for instance as measured by parameters such as oxidative stability, cetane number, oil stability index (OSI), Iodine value, and APE/BAPE index. Methods for measuring such parameters are well known in the art (e.g. Knothe, 2002).

Soybean oil in its commercially available unrefined or refined, edible-grade state, is a fairly stable and slow-drying oil. Soybean oil can also be modified to enhance its reactivity under ambient conditions or, with the input of energy in various forms, to cause the oil to copolymerize or cure to a dry film. Some of these forms of modification have included epoxidation, alcoholysis or transesterification, direct esterification, metathesis, isomerization, monomer modification, and various forms of polymerization, including heat bodying. The reactive linolenic-acid component of soybean oil with its double bonds may be more useful than the predominant oleic- and linoleic-acid components for many industrial uses.

Solvents can also be prepared using soy-based ingredients. For example, methyl soyate, a soybean-oil based methyl ester, is gaining market acceptance as an excellent solvent replacement alternative in applications such as parts cleaning and degreasing, paint and ink removal, and oil spill remediation. It is also being marketed in numerous formulated consumer products including hand cleaners, car waxes and graffiti removers. Methyl soyate is produced by the transesterification of soybean oil with methanol. It is commercially available from numerous manufacturers and suppliers. As a solvent, methyl soyate has important environmental- and safety-related properties that make it attractive for industrial applications. It is lower in toxicity than most other solvents, is readily biodegradable, and has a very high flash point and a low level of volatile organic compounds (VOCs). The compatibility of methyl soyate is excellent with metals, plastics, most elastomers and other organic solvents. Current uses of methyl soyate include cleaners, paint strippers, oil spill cleanup and bioremediation, pesticide adjuvants, corrosion preventives and biodiesel fuels additives.

Further, this invention provides a method of producing an oil product comprising: (a) obtaining a soybean seed of the invention (b) planting and growing said seed into a mature plant (c) harvesting seed from the plant (d) preparing an oil product from the harvested seed. In certain embodiments of the invention, the oil product may comprise food oil, feed, fuel, resins, disinfectants, fungicides, rubber, fuel, paint, cosmetics, pharmaceuticals, inks and lubricants. Examples of food oils are cooking oil, emulsified products (e.g. mayonnaise, shortening, margarine, and salad dressing), intermediate moisture foods (e.g. dog foods).

In addition, this invention provides a method of producing a protein product comprising: (a) obtaining a soybean seed of the invention, (b) planting and growing the seed into a mature plant, (c) harvesting seed from the plant, and (d) preparing a protein product from the harvested seed. In certain embodiments of the invention, the protein product may comprise protein isolate, meal, flour or soybean hulls for food and feed.

In addition, this invention provides a method of producing a food or feed product comprising: (a) obtaining a soybean seed of the invention, (b) planting and growing said seed into a mature plant, (c) harvesting seed from the plant, and (d) preparing a food or feed product from the harvested seed. In certain embodiments of the invention, the food or feed products may comprise animal feed, pharmaceuticals, soy milk, tofu, roasted soybeans, baby foods, soynut butter, and other soy derivatives.

This invention provides a method of producing an industrial product comprising: (a) obtaining a soybean seed of the invention, (b) planting and growing said seed into a mature plant, (c) harvesting seed from said plant, and (d) preparing an industrial product from said harvested seed. In certain embodiments of the invention, the industrial product may comprise a fuel, lubricant, resin, binder, glue, adhesive, ink, paint, fungicide, disinfectant, rubber, cosmetic, caulking compound, wallboard, anti-foam agent, alcohol, wax, solvent, or film.

In still another aspect of the invention there is provided a method for producing soybean seed, comprising crossing the plant of the invention with itself or with a second soybean plant. This, this method may comprise preparing a hybrid soybean seed by crossing a plant of the invention with a second, distinct, soybean plant.

It is further understood that a soybean plant of the present invention may exhibit the characteristics of any maturity group. The pollen from the selected soybean plant can be cryopreserved and used in crosses with elite lines from other maturity groups to introgress a the fungal disease resistance locus into a line that would not normally be available for crossing in nature. Pollen cryopreservation techniques are well known in the art (e.g. Liang et al. 1993; Honda et al. 2002; Tyagi and Hymowitz 2003).

Soybean seed oil levels are highly impacted by environment. Oil concentration increases with decreasing latitude, therefore, soybeans in early maturity group soybeans (00-I) generally have lower oil levels than later maturing soybeans (e.g. Yaklich et al. 2002). The decrease in oil concentrations is attributed to lower temperatures and shorter growing season (Piper and Boote, 1999). In addition, soybeans cultivated under drought stress tend to produce seeds with decreased protein and increased oil (Specht et al. 2001).

Plants of the present invention can be part of or generated from a breeding program. The choice of breeding method depends on the mode of plant reproduction, the heritability of the trait(s) being improved, and the type of cultivar used commercially (e.g., F₁ hybrid cultivar, pure line cultivar, etc). A cultivar is a race or variety of a plant that has been created or selected intentionally and maintained through cultivation.

Selected, non-limiting approaches for breeding the plants of the present invention are set forth below. A breeding program can be enhanced using marker assisted selection (MAS) of the progeny of any cross. It is further understood that any commercial and non-commercial cultivars can be utilized in a breeding program. Factors such as, for example, emergence vigor, vegetative vigor, stress tolerance, disease resistance, branching, flowering, seed set, seed size, seed density, standability, and threshability etc. will generally dictate the choice.

For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection. In a preferred embodiment a backcross or recurrent breeding program is undertaken.

The complexity of inheritance influences choice of the breeding method. Backcross breeding can be used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes. The use of recurrent selection in self-pollinating crops depends on the ease of pollination, the frequency of successful hybrids from each pollination event, and the number of hybrid offspring from each successful cross.

Breeding lines can be tested and compared to appropriate standards in environments representative of the commercial target area(s) for two or more generations. The best lines are candidates for new commercial cultivars; those still deficient in traits may be used as parents to produce new populations for further selection.

One method of identifying a superior plant is to observe its performance relative to other experimental plants and to a widely grown standard cultivar. If a single observation is inconclusive, replicated observations can provide a better estimate of its genetic worth. A breeder can select and cross two or more parental lines, followed by repeated self-pollinating and selection, producing many new genetic combinations.

New soybean varieties may be developed by crossing elite varieties and selecting of superior progeny from the hybrid crosses. The hybrid seed can be produced by manual crosses between selected male-fertile parents or by using male sterility systems. Hybrids are selected for certain single gene traits such as pod color, flower color, seed yield, pubescence color or herbicide resistance which indicate that the seed is truly a hybrid. Additional data on parental lines, as well as the phenotype of the hybrid, influence the breeder's decision whether to continue with the specific hybrid cross.

Pedigree breeding and recurrent selection breeding methods can be used to develop cultivars from breeding populations. Breeding programs combine desirable traits from two or more cultivars or various broad-based sources into breeding pools from which cultivars are developed by self-pollinating and selection of desired phenotypes. New cultivars can be evaluated to determine which have commercial potential.

Pedigree breeding is used commonly for the improvement of self-pollinating crops. Two parents which possess favorable, complementary traits are crossed to produce an F₁. An F₂ population is produced by self-pollinating one or several F₁'s. Selection of the best individuals in the best families is selected. Replicated testing of families can begin in the F₄ generation to improve the effectiveness of selection for traits with low heritability. At an advanced stage of inbreeding (i.e., F₆ and F₇), the best lines or mixtures of phenotypically similar lines are tested for potential release as new cultivars.

Backcross breeding has been used to transfer genes for a simply inherited, highly heritable trait into a desirable homozygous cultivar or inbred line, which is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting parent is expected to have the attributes of the recurrent parent (e.g., cultivar) and the desirable trait transferred from the donor parent.

The single-seed descent procedure in the strict sense refers to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advance is completed.

In a multiple-seed procedure, soybean breeders commonly harvest one or more pods from each plant in a population and thresh them together to form a bulk. Part of the bulk is used to plant the next generation and part is put in reserve. The procedure has been referred to as modified single-seed descent or the pod-bulk technique.

The multiple-seed procedure has been used to save labor at harvest. It is considerably faster to thresh pods with a machine than to remove one seed from each by hand for the single-seed procedure. The multiple-seed procedure also makes it possible to plant the same number of seed of a population each generation of advancement.

Descriptions of other breeding methods that are commonly used for different traits and crops can be found in one of several reference books (e.g. Fehr 1987).

Through breeding techniques, improved seed oil levels can be combined with any other desirable seed or agronomic trait. The present invention provides high oil Glycine max plants, including transgenic plants, that contain one or more genes for herbicide tolerance, increased yield, insect control, fungal disease resistance, virus resistance, nematode resistance, bacterial disease resistance, mycoplasma disease resistance, modified oils production, high oil production, high protein production, germination and seedling growth control, enhanced animal and human nutrition, low raffinose, environmental stress resistance, altered morphological characteristics, increased digestibility, industrial enzymes, pharmaceutical proteins, peptides and small molecules, improved processing traits, improved flavor, nitrogen fixation, hybrid seed production, reduced allergenicity, production of biopolymers, and biofuels among others. These agronomic traits can be provided by the methods of plant biotechnology as transgenes in Glycine max.

The present invention also provides for parts of the plants of the present invention. Plant parts, without limitation, include seed, endosperm, ovule and pollen. In a particularly preferred embodiment of the present invention, the plant part is a seed.

Plants or parts thereof of the present invention may be grown in culture and regenerated. Methods for the regeneration of Glycine max plants from various tissue types and methods for the tissue culture of Glycine max are known in the art (see, for example, Widholm et al. (1996). Regeneration techniques for plants such as Glycine max can use as the starting material a variety of tissue or cell types. With Glycine max in particular, regeneration processes have been developed that begin with certain differentiated tissue types such as meristems (Cartha et al. 1981), hypocotyl sections (Cameya et al. 1981), and stem node segments (Saka et al. 1980; Cheng et al. 1980). Regeneration of whole sexually mature Glycine max plants from somatic embryos developed from explants of immature Glycine max embryos has been reported (e.g. Ranch et al. 1985). Regeneration of mature Glycine max plants from tissue culture by organogenesis and embryogenesis has also been reported (Barwale et al. 1986; Wright et al. 1986).

The definitions and methods provided define the present invention and guide those of ordinary skill in the art in the practice of the present invention. Unless otherwise noted, terms are to be understood according to conventional usage by those of ordinary skill in the relevant art.

As used herein, “soybean” refers to the species Glycine max, Glycine soja or any species that is sexually compatible with Glycine max.

As used herein, “oil” refers to any hydrophobic liquid.

As used herein, “vegetable oil” refers to any hydrophobic liquid derived from plants.

As used herein, “meal” refers to the remains of the seed after the oil has been extracted.

As used herein, a “high oil seed” or “elevated oil seed” refers to seed with greater than 23% oil calculated on a dry weight basis.

As used herein, a “high oil soybean” refers to soybean plant that produces a high oil seed.

As used herein, a “high oil genes” refers to traits or genes conferring high oil in a soybean seed.

As used herein, a “trait” refers to an observable and/or measurable characteristic of an organism, such as a trait of a plant, for example, tolerance to an herbicide, insect and microbe.

As used herein, a “transgene” refers to a foreign gene that is placed into an organism by the process of plant transformation.

As used herein, a “foreign gene” refers to any nucleic acid that is introduced into the genome of an organism by experimental manipulations and may include gene sequences found in that organism by experimental manipulations and may include gene sequences found in that organism.

As used herein, NIT (near-infrared transmission) is a technique that can determine protein, oil, moisture, starch, lipids, and cellulose in oilseeds.

As used herein, “M₀” and “mother line” refers to generation of seed treated with a mutagenic agent. Mutagenic agents can be, but are not limited to radiation, such as x-rays, neutrons, gamma rays, ultraviolet and laser beams, and chemical mutagens, such as ethyl methane sulfonate.

As used herein, “M₁” refers to the first generation of plants after treatment with a mutagen. An “M₂” population is produced by self-pollinating one or several M₁'s. The best individuals in the best families are selected to carry forward to the next generation.

As used herein, “line” refers to a group of individual plants from the similar parentage with similar traits. An “elite line” is any line that has resulted from breeding and selection for superior agronomic performance. Additionally, an elite line is sufficiently homogenous and homozygous to be used for commercial production. Elite lines may be used in the further breeding efforts to develop new elite lines.

### EXAMPLES

### Example 1

### Creation of Mutant Genes Conferring Elevated Oil Levels in Seed

Seed quality, such as oil levels and oil quality or composition, is a focus for many soybean breeding programs. Oil levels in soybean seed has typically been increased through traditional breeding efforts, such as mutation breeding, which can increase the genetic variability within soybean and can be used to develop and discover novel genes to elevate oil levels in the seed.

Six high oil soybean lines were selected to undergo mutagenesis to increase oil levels. Three pounds of seed (˜10,000 seeds) was exposed to 20 Krads/hr of gamma ray radiation for each mother line. Following radiation, all M₁ seeds were planted. From each surviving M₁ plant, one pod was harvested and all pods were bulked. M₂ seeds were planted and each M₂ plant was harvested individually. Oil content was evaluated for each plant by NIT (Tables 1-2). Plants were selected that had oil content greater than three standard deviations from the mother line.

TABLE 1
Comparison of oil levels (% on dry weight basis) and protein levels
(% on dry weight basis) between mother line (MV0026 or MV0027)
and high oil mutants across generations.

| | M₁ | | M₂ | | M_{2:3} | | M_{2:4} | |
|---|---|---|---|---|---|---|---|---|
| | Oil | Protein | Oil | Protein | Oil | Protein | Oil | Protein |
| Mother line: MV0026 | 23 | 38.3 | 25 | 37.2 | 21.4 | 40.6 | 24 | 38.3 |
| MV0026-3166 | 25 | 35.9 | 27 | 35 | 24.1 | 36.3 | 25.8 | 35.1 |
| MV0026-1758 | 25 | 37.4 | 27 | 35.3 | 23.5 | 38 | 25.4 | 36.1 |
| MV0026-3568 | 25 | 36.8 | 26 | 35.9 | 23.7 | 37.7 | 25.4 | 36.3 |
| MV0026-4338 | 25 | 36.8 | 26 | 35.7 | 23.7 | 36.8 | 25.2 | 36.4 |
| MV0026-0123 | 25 | 37.2 | 26 | 35.4 | 23.8 | 36.8 | 25.2 | 36 |
| MV0026-5018 | 26 | 36.7 | 25 | 39 | 23.8 | 39 | 25.2 | 36 |
| Mother line: MV0027 | | | 21.7 | 42.5 | 19.8 | 44.8 | 21.1 | 43.6 |
| M₁: MV0027-3947 | | | | | 234 | 36.8 | 24.9 | 34.9 |
| M₁: MV0027-2535 | | | 25.9 | 29.3 | — | — | 24.4 | 32.9 |
| M₁: MV0027-2480 | | | 26.4 | 28.3 | — | — | 24.1 | 33.6 |

The selected M₃ seed was planted. Each M_{3:3} plot was harvested individually. Oil content was evaluated for each plot by NIT. M_{2:3} lines with oil significantly (P<0.05) higher than oil of the mother line were selected. M_{3:3} lines were planted in single row plots. Each M_{3:4} plot was harvested individually and oil content was evaluated by NIT. Lines with oil significantly (p<0.05) higher than oil of the mother line were identified as high oil mutants. The increase in oil level ranged from 1-4% at the M_{2:4} generation. Tables 3 and 4 illustrate that oil levels increased in selected mutants without a significant impact on yield.

TABLE 2
Comparison of oil levels (% on dry weight basis) and protein levels
(% on dry weight basis) between the mother line and F_{3:4}
generation of high oil and protein mutants.

| | | Percent Oil | | | Percent Protein | | |
|---|---|---|---|---|---|---|---|
| Soybean | | (DWB) | | | (DWB) | | |
| Line | Type | Mean | Δ | LSD | Mean | Δ | LSD |
| MV0026 | Mother | 23.967 | — | 0.361 | 38.307 | — | 0.604 |
| MV0026- | Mutant | 25.750 | 1.783 | 0.361 | 35.097 | −3.210 | 0.604 |
| 3166 | | | | | | | |
| MV0026- | Mutant | 25.407 | 1.440 | 0.361 | 36.118 | −2.188 | 0.604 |
| 1758 | | | | | | | |
| MV0026- | Mutant | 25.400 | 1.433 | 0.361 | 36.253 | −2.053 | 0.604 |
| 3568 | | | | | | | |
| MV0026- | Mutant | 25.247 | 1.280 | 0.361 | 36.367 | −1.940 | 0.604 |
| 4338 | | | | | | | |
| MV0026- | Mutant | 25.200 | 1.233 | 0.361 | 36.020 | −2.287 | 0.604 |
| 0123 | | | | | | | |
| MV0026- | Mutant | 25.150 | 1.183 | 0.361 | 37.410 | −0.897 | 0.604 |
| 5018 | | | | | | | |
| MV0040 | Mother | 19.627 | — | 0.268 | 42.600 | — | 0.478 |
| MV0040- | Mutant | 19.967 | 0.340 | 0.268 | 42.960 | 0.360 | 0.478 |
| 4313 | | | | | | | |
| MV0040- | Mutant | 19.907 | 0.280 | 0.268 | 42.513 | −0.087 | 0.478 |
| 0689 | | | | | | | |
| MV0040- | Mutant | 19.682 | 0.055 | 0.268 | 42.926 | 0.326 | 0.478 |
| 0497 | | | | | | | |
| MV0040- | Mutant | 19.133 | −0.493 | 0.268 | 44.267 | 1.667 | 0.478 |
| 3346 | | | | | | | |
| MV0040- | Mutant | 19.100 | −0.527 | 0.268 | 43.933 | 1.333 | 0.478 |
| 1698 | | | | | | | |
| MV0040- | Mutant | 19.047 | −0.580 | 0.268 | 44.287 | 1.687 | 0.478 |
| 3711 | | | | | | | |
| MV0040- | Mutant | 18.993 | −0.633 | 0.268 | 44.207 | 1.607 | 0.478 |
| 3641 | | | | | | | |
| MV0040- | Mutant | 18.606 | −1.020 | 0.268 | 44.819 | 2.219 | 0.478 |
| 1379 | | | | | | | |
| MV0040- | Mutant | 18.406 | −1.220 | 0.268 | 44.730 | 2.130 | 0.478 |
| 2846 | | | | | | | |
| MV0040- | Mutant | 18.259 | −1.368 | 0.268 | 44.925 | 2.325 | 0.478 |
| 3389 | | | | | | | |
| MV0041 | Mother | 22.217 | — | 0.268 | 39.762 | — | 0.478 |
| MV0041- | Mutant | 23.040 | 0.823 | 0.268 | 37.447 | −2.315 | 0.478 |
| 2695 | | | | | | | |
| MV0041- | Mutant | 23.007 | 0.789 | 0.268 | 38.033 | −1.729 | 0.478 |
| 7308 | | | | | | | |
| MV0041- | Mutant | 22.047 | −0.171 | 0.268 | 41.147 | 1.385 | 0.478 |
| 2770 | | | | | | | |
| MV0041- | Mutant | 21.799 | −0.418 | 0.268 | 40.698 | 0.936 | 0.478 |
| 4232 | | | | | | | |
| MV0041- | Mutant | 19.289 | −2.929 | 0.268 | 43.505 | 3.743 | 0.478 |
| 3858 | | | | | | | |
| MV0041- | Mutant | 18.849 | −3.368 | 0.268 | 44.640 | 4.878 | 0.478 |
| 2759 | | | | | | | |
| MV0043 | Mother | 21.087 | — | 0.216 | 43.573 | — | 0.498 |
| MV0043- | Mutant | 24.853 | 3.767 | 0.216 | 34.920 | −8.653 | 0.498 |
| 3947 | | | | | | | |
| MV0027- | Mutant | 24.387 | 3.300 | 0.216 | 32.914 | −10.660 | 0.498 |
| 2535 | | | | | | | |
| MV0027- | Mutant | 24.087 | 3.000 | 0.216 | 33.627 | −9.947 | 0.498 |
| 2480 | | | | | | | |
| MV0043- | Mutant | 21.820 | 0.733 | 0.216 | 42.840 | −0.733 | 0.498 |
| 2226 | | | | | | | |
| MV0043- | Mutant | 21.793 | 0.707 | 0.216 | 42.753 | −0.820 | 0.498 |
| 0146 | | | | | | | |
| MV0043- | Mutant | 21.660 | 0.573 | 0.216 | 42.960 | −0.613 | 0.498 |
| 4359 | | | | | | | |
| MV0043- | Mutant | 21.660 | 0.573 | 0.216 | 43.107 | −0.467 | 0.498 |
| 3397 | | | | | | | |
| MV0043- | Mutant | 21.653 | 0.567 | 0.216 | 43.160 | −0.413 | 0.498 |
| 0209 | | | | | | | |
| MV0043- | Mutant | 21.613 | 0.527 | 0.216 | 42.933 | −0.640 | 0.498 |
| 0122 | | | | | | | |
| MV0043- | Mutant | 21.594 | 0.507 | 0.216 | 43.006 | −0.567 | 0.498 |
| 3363 | | | | | | | |
| MV0043- | Mutant | 21.543 | 0.456 | 0.216 | 44.064 | 0.491 | 0.498 |
| 2162 | | | | | | | |
| MV0043- | Mutant | 21.540 | 0.453 | 0.216 | 43.693 | 0.120 | 0.498 |
| 0589 | | | | | | | |
| MV0043- | Mutant | 21.507 | 0.420 | 0.216 | 43.480 | −0.093 | 0.498 |
| 2389 | | | | | | | |
| MV0043- | Mutant | 21.473 | 0.387 | 0.216 | 43.300 | −0.273 | 0.498 |
| 4175 | | | | | | | |
| MV0043- | Mutant | 21.451 | 0.364 | 0.216 | 43.485 | −0.088 | 0.498 |
| 3456 | | | | | | | |
| MV0043- | Mutant | 21.359 | 0.272 | 0.216 | 43.660 | 0.087 | 0.498 |
| 0336 | | | | | | | |
| MV0043- | Mutant | 21.253 | 0.167 | 0.216 | 43.973 | 0.400 | 0.498 |
| 1059 | | | | | | | |
| MV0043- | Mutant | 21.207 | 0.120 | 0.216 | 43.893 | 0.320 | 0.498 |
| 2136 | | | | | | | |
| MV0043- | Mutant | 20.259 | −0.828 | 0.216 | 44.710 | 1.137 | 0.498 |
| 2298 | | | | | | | |
| MV0043- | Mutant | 20.202 | −0.885 | 0.216 | 45.517 | 1.944 | 0.498 |
| 0313 | | | | | | | |
| MV0043- | Mutant | 20.127 | −0.960 | 0.216 | 44.900 | 1.327 | 0.498 |
| 4492 | | | | | | | |
| MV0042 | Mother | 21.462 | — | 0.248 | 41.934 | — | 0.495 |
| MV0042- | Mutant | 22.000 | 0.538 | 0.248 | 41.860 | −0.074 | 0.495 |
| 5237 | | | | | | | |
| MV0042- | Mutant | 21.947 | 0.484 | 0.248 | 40.473 | −1.461 | 0.495 |
| 9098 | | | | | | | |
| MV0042- | Mutant | 21.946 | 0.483 | 0.248 | 40.882 | −1.052 | 0.495 |
| 8829 | | | | | | | |
| MV0042- | Mutant | 21.934 | 0.471 | 0.248 | 40.649 | −1.286 | 0.495 |
| 7710 | | | | | | | |
| MV0042- | Mutant | 21.893 | 0.431 | 0.248 | 40.827 | −1.108 | 0.495 |
| 0510 | | | | | | | |
| MV0042- | Mutant | 21.873 | 0.411 | 0.248 | 40.720 | −1.214 | 0.495 |
| 5256 | | | | | | | |
| MV0042- | Mutant | 21.847 | 0.384 | 0.248 | 41.713 | −0.221 | 0.495 |
| 0582 | | | | | | | |
| MV0042- | Mutant | 21.767 | 0.304 | 0.248 | 42.073 | 0.139 | 0.495 |
| 3650 | | | | | | | |
| MV0042- | Mutant | 21.667 | 0.204 | 0.248 | 42.313 | 0.379 | 0.495 |
| 8027 | | | | | | | |
| MV0042- | Mutant | 20.897 | −0.566 | 0.248 | 43.646 | 1.712 | 0.495 |
| 0436 | | | | | | | |
| MV0042- | Mutant | 20.753 | −0.709 | 0.248 | 43.153 | 1.219 | 0.495 |
| 8624 | | | | | | | |
| MV0042- | Mutant | 20.660 | −0.802 | 0.248 | 43.300 | 1.366 | 0.495 |
| 9207 | | | | | | | |
| MV0042- | Mutant | 20.227 | −1.236 | 0.248 | 44.607 | 2.672 | 0.495 |
| 2981 | | | | | | | |
| MV0042- | Mutant | 19.767 | −1.696 | 0.248 | 44.146 | 2.212 | 0.495 |
| 2666 | | | | | | | |
| MV0042- | Mutant | 19.167 | −2.296 | 0.248 | 44.227 | 2.292 | 0.495 |
| 9038 | | | | | | | |
| MV0042- | Mutant | 18.768 | −2.694 | 0.248 | 45.410 | 3.476 | 0.495 |
| 6609 | | | | | | | |

TABLE 3
Comparison of oil level (% on dry weight basis) and yield between high
oil mutants and the mother line (M₀) across three environments.

| | Percent oil | Yield | Lbs. of |
|---|---|---|---|
| Line | (DWB) | (Bu/acre) | Oil/Acre |
| MV0026-3166 | 25.01 * | 43.31 | 14.62 |
| MV0026-3568 | 24.84 * | 43.69 | 14.52 |
| MV0026-4338 | 24.84 * | 45.15 | 14.52 |
| MV0026-0123 | 24.78 * | 41.4 | 14.48 |
| MV0026-1758 | 24.60 * | 44.72 | 14.38 |
| M₀: MV0026 | 23.64 | 42.56 | 13.81 |

Oil content of mutants with * were significantly (p < 0.05) higher than oil content of mother line.

TABLE 4
Comparison of oil level (% on dry weight basis) between
mutants and the mother line across three environments.

| | Mean | Std Dev | Min | Max |
|---|---|---|---|---|
| Line | (Oil Wt %) | (Oil Wt %) | (Oil Wt %) | (Oil Wt %) |
| MV0030 | 21.93 | 1.58 | 17.57 | 24.47 |
| MV0028 | 22.47 | 0.86 | 20.41 | 24.5 |
| MT001 | 29.27 | 0.87 | 27.38 | 30.82 |
| MT002 | 28.29 | 1.14 | 25.84 | 30.44 |
| MT003 | 28.25 | 1.59 | 25.37 | 30.69 |
| MT004 | 28.47 | 0.86 | 26.36 | 30.11 |
| MT005 | 27.58 | 1.98 | 23.22 | 30.72 |
| MT006 | 27.74 | 1.28 | 25.31 | 30.11 |
| MT007 | 28.16 | 1.38 | 25.84 | 31.46 |
| MT008 | 27.89 | 1.29 | 24.24 | 30.44 |
| MT009 | 27.79 | 1.57 | 25.04 | 30.89 |
| MT010 | 28.35 | 1.23 | 26.17 | 30.47 |
| MT011 | 20.09 | 1.27 | 16.54 | 22.78 |
| MT012 | 21.59 | 1.41 | 18.64 | 23.93 |
| MT013 | 23.55 | 1.6 | 21.02 | 26.95 |

The oil mutations did not cause shifts in fatty acid or carbohydrate composition within the seed (Table 5). Seeds of six high oil mutants from cycle 1 radiation, MV0026-3166, MV0026-3568, MV0026-4338, MV0026-0123, MV0026-1758, and MV0026-5018, and their mother line MV0026 were sampled for composition analysis. Each soybean line was evaluated for fatty acid composition (palmitate, stearate, oleate, linolenate and linolenate) and carbohydrate composition (sucrose, raffinose, and stachyose). The fatty acids and carbohydrates levels in high oil mutants were within normal ranges for soybean.

TABLE 5
Fatty acids and carbohydrates composition
of high oil mutants and mother lines.

| | | M₀: | M₁: | M₀: | M₁: |
|---|---|---|---|---|---|
| | | MV0026 | MV0026 | MV0027 | MV0027 |
| Palmitate | Mean | 9.1 | 9.5 | 9.4 | 9.7 |
| | Max | 9.4 | 10 | 9.7 | 10.2 |
| | Min | 8.8 | 9.1 | 9.3 | 9.2 |
| Stearate | Mean | 3.9 | 3.8 | 4.4 | 4.3 |
| | Max | 4 | 4.2 | 4.7 | 4.7 |
| | Min | 3.8 | 3.5 | 4.1 | 3.9 |
| Oleate | Mean | 22.2 | 22.3 | 25.9 | 24.8 |
| | Max | 22.4 | 24.7 | 27.1 | 25.9 |
| | Min | 21.7 | 21.1 | 24.6 | 23.8 |
| Linoleate | Mean | 55.4 | 56.8 | 52.6 | 52.9 |
| | Max | 56.2 | 59.1 | 53.9 | 53.7 |
| | Min | 54.4 | 54.2 | 51.3 | 52.1 |
| Linolenate | Mean | 6.8 | 6.4 | 7 | 7.3 |
| | Max | 7.3 | 6.7 | 7.1 | 7.6 |
| | Min | 6.5 | 5.8 | 6.7 | 7.1 |
| Sucrose | Mean | 5.1 | 5.3 | 4.1 | 6 |
| | Max | 5.5 | 5.9 | 4.4 | 6.9 |
| | Min | 4.8 | 4.5 | 3.9 | 4.8 |
| Raffinose | Mean | 0.9 | 1.2 | 0.8 | 0.8 |
| | Max | 1 | 1.3 | 0.9 | 1 |
| | Min | 0.9 | 1 | 0.7 | 0.7 |
| Stachyose | Mean | 3.5 | 3.8 | 4.1 | 4.2 |
| | Max | 3.6 | 4.1 | 4.3 | 4.6 |
| | Min | 3.5 | 3.4 | 4 | 3.7 |

### Example 2

### Elevated Oil Levels in High Oil Mutants with Additional Cycles of Mutagenesis

Seed oil levels were further increased by conducting a second cycle of radiation. A mother line was selected for the high oil trait from the mutants generated in the first cycle of radiation. Mother line MV0026-3166,5018 was formed by bulking two high oil mutants, MV0026-3166 and MV0026-5018, from the initial cycle of radiation. Additionally, MV0026-4126 was also selected from the first radiation cycle. Seeds of MV0026-3166,5018 and MV0026-4126 were exposed to 20 Krads/hr of gamma ray radiation. The increase in oil level ranged from 0.9-2.0%, with MV0026-4126-0692 having 29.2% oil (Tables 6-8). A stepwise increase in oil was observed from the first and second cycle of radiation. Oil levels increase 3% within the seed from the two cycles of radiation. The initial cycle of radiation increased oil levels 1% from the mother line, while the second cycle of radiation increased oil levels an additional 2%. Additional cycles of mutagenesis can be conducted to further increase oil content of seed.

TABLE 6
Comparison of seed oil and protein levels between
high oil mutants obtained from second cycle of radiation
and M₀ mother line MV0026-3166, 5018.

| | M₂ | | M₃ | |
|---|---|---|---|---|
| Mother line and | Oil | Protein | Oil | Protein |
| Mutant's | (%) | (%) | (%) | (%) |
| Mother line: | | | | |
| MV0026-3166, 5018 | 22.7 | 40.2 | 25.2 | 36.9 |
| MV0026-3166, 5018-2374 | 24.7 | 35.7 | 27.2 | 32.4 |
| MV0026-3166, 5018-9450 | 24 | 38.3 | 26.5 | 34.6 |
| MV0026-3166, 5018-2605 | 23.8 | 37.6 | 26.3 | 34.6 |
| MV0026-3166, 5018-2579 | 23.6 | 38 | 26.1 | 35 |

TABLE 7
Comparison oil levels and protein levels for high oil
mutants obtained from second cycle of radiation.

| | Oil | Protein |
|---|---|---|
| High Oil Line | (%) | (%) |
| MV0026-4126-0692 | 29.2 | 28.1 |
| MV0026-4126-0156 | 28.8 | 28.6 |
| MV0026-4126-0126 | 28.6 | 28.9 |
| MV0026-4126-0216 | 28.6 | 29.9 |
| MV0026-4126-0089 | 28.5 | 28.5 |
| MV0026-4126-0295 | 28.5 | 29.8 |
| MV0026-4126-0111 | 28.3 | 29.3 |
| MV0026-4126-4613 | 28.3 | 29 |
| MV0026-4126-0094 | 28.2 | 29.9 |
| MV0026-4126-0058 | 28.2 | 29.7 |
| MV0026-4126-0051 | 28.2 | 29.8 |
| MV0026-4126-0128 | 28.2 | 29.4 |
| MV0026-4126-0304 | 28.2 | 29.5 |
| MV0026-4126-5411 | 28.2 | 29.3 |
| MV0026-4126-0212 | 28.1 | 29.7 |

TABLE 8
Comparison oil levels and protein levels for high oil mutants obtained
from second cycle of radiation and mother lines.

| | | Percent Oil | | | Percent Protein | | |
|---|---|---|---|---|---|---|---|
| | | (DWB) | | | (DWB) | | |
| Soybean Line | Type | Mean | Δ | LSD | Mean | Δ | LSD |
| MV0026 | Mother | 23.967 | — | — | 38.307 | — | — |
| MV0026-[3166,5018] | Mother | 25.373 | — | — | 36.707 | — | — |
| MV0026-[3166,5018]-2374 | Mutant | 27.224 | 1.850 | 0.361 | 32.399 | −4.307 | 0.604 |
| MV0026-[3166,5018]-2605 | Mutant | 26.267 | 0.893 | 0.361 | 34.571 | −2.136 | 0.604 |
| MV0026-[3166,5018]-2579 | Mutant | 26.140 | 0.767 | 0.361 | 34.967 | −1.740 | 0.604 |
| MV0026-[3166,5018]-2231 | Mutant | 25.587 | 0.213 | 0.361 | 36.880 | 0.173 | 0.604 |
| CBN1900H0 | Mother | 23.840 | — | — | 38.100 | — | — |
| MV0026-[3166,5018] | Mother | 25.093 | — | — | 37.160 | — | — |
| MV0026-[3166,5018]-3439 | Mutant | 25.974 | 0.881 | 0.346 | 34.583 | −2.577 | 0.574 |
| MV0026-[3166,5018]-3301 | Mutant | 25.913 | 0.820 | 0.346 | 34.860 | −2.300 | 0.574 |
| MV0026-[3166,5018]-3725 | Mutant | 25.900 | 0.806 | 0.346 | 34.498 | −2.662 | 0.574 |
| MV0026-[3166,5018]-3510 | Mutant | 25.840 | 0.747 | 0.346 | 35.093 | −2.067 | 0.574 |
| MV0026-[3166,5018]-2723 | Mutant | 25.753 | 0.660 | 0.346 | 35.040 | −2.120 | 0.574 |
| MV0026-[3166,5018]-3504 | Mutant | 25.753 | 0.660 | 0.346 | 35.233 | −1.927 | 0.574 |
| MV0026-[3166,5018]-3468 | Mutant | 25.715 | 0.622 | 0.346 | 35.376 | −1.784 | 0.574 |
| MV0026-[3166,5018]-2957 | Mutant | 25.713 | 0.620 | 0.346 | 35.187 | −1.973 | 0.574 |
| MV0026-[3166,5018]-3507 | Mutant | 25.680 | 0.587 | 0.346 | 35.227 | −1.933 | 0.574 |
| MV0026-[3166,5018]-3511 | Mutant | 25.673 | 0.580 | 0.346 | 35.240 | −1.920 | 0.574 |
| MV0026 | Mother | 23.47 | — | — | 38.33 | — | — |
| MV0026-41260001 | Mother | 25.63 | — | — | 34.30 | — | — |
| MV0026-4126-0111 | Mutant | 28.01 | 2.38 | 0.760 | 29.28 | −5.02 | 1.536 |
| MV0026-4126-0156 | Mutant | 26.99 | 1.36 | 0.760 | 31.00 | −3.30 | 1.536 |
| MV0026-4126-0128 | Mutant | 26.94 | 1.31 | 0.760 | 30.83 | −3.47 | 1.536 |
| MV0026-4126-0051 | Mutant | 26.93 | 1.30 | 0.760 | 30.88 | −3.42 | 1.536 |
| MV0026-4126-0075 | Mutant | 26.93 | 1.30 | 0.760 | 31.56 | −2.74 | 1.536 |
| MV0026-4126-0126 | Mutant | 26.80 | 1.17 | 0.760 | 31.54 | −2.76 | 1.536 |
| MV0026-4126-0103 | Mutant | 26.78 | 1.15 | 0.760 | 31.73 | −2.57 | 1.536 |
| MV0026-4126-0074 | Mutant | 26.75 | 1.12 | 0.760 | 32.72 | −1.58 | 1.536 |
| MV0026-4126-0108 | Mutant | 26.56 | 0.93 | 0.760 | 32.04 | −2.26 | 1.536 |
| MV0026-4126-0144 | Mutant | 26.54 | 0.91 | 0.760 | 31.58 | −2.72 | 1.536 |
| MV0026-4126-0082 | Mutant | 26.52 | 0.89 | 0.760 | 32.29 | −2.01 | 1.536 |
| MV0026-4126-0094 | Mutant | 26.48 | 0.85 | 0.760 | 32.00 | −2.30 | 1.536 |
| MV0026-4126-0056 | Mutant | 26.40 | 0.77 | 0.760 | 32.04 | −2.26 | 1.536 |
| MV0026-4126-0125 | Mutant | 26.40 | 0.77 | 0.760 | 32.44 | −1.86 | 1.536 |
| MV0026 | Mother | 23.01 | — | — | 39.31 | — | — |
| MV0026-41260001 | Mother | 24.77 | — | — | 35.09 | — | — |
| MV0026-4126-0261 | Mutant | 26.91 | 2.14 | 0.760 | 31.84 | −3.25 | 1.536 |
| MV0026-4126-1462 | Mutant | 26.85 | 2.08 | 0.760 | 32.06 | −3.03 | 1.536 |
| MV0026-4126-0377 | Mutant | 26.80 | 2.03 | 0.760 | 32.12 | −2.97 | 1.536 |
| MV0026-4126-0692 | Mutant | 26.76 | 1.99 | 0.760 | 31.81 | −3.28 | 1.536 |
| MV0026-4126-0295 | Mutant | 26.29 | 1.52 | 0.760 | 32.99 | −2.10 | 1.536 |
| MV0026-4126-0304 | Mutant | 26.01 | 1.24 | 0.760 | 33.03 | −2.06 | 1.536 |
| MV0026-4126-0216 | Mutant | 25.96 | 1.19 | 0.760 | 34.35 | −0.74 | 1.536 |
| MV0026-4126-1470 | Mutant | 25.92 | 1.15 | 0.760 | 33.29 | −1.80 | 1.536 |
| MV0026-4126-0373 | Mutant | 25.89 | 1.12 | 0.760 | 33.33 | −1.76 | 1.536 |
| MV0026-4126-1202 | Mutant | 25.86 | 1.09 | 0.760 | 33.18 | −1.91 | 1.536 |
| MV0026-4126-0473 | Mutant | 25.83 | 1.06 | 0.760 | 33.67 | −1.42 | 1.536 |
| MV0026-4126-0334 | Mutant | 25.79 | 1.02 | 0.760 | 33.46 | −1.63 | 1.536 |
| MV0026-4126-1472 | Mutant | 25.79 | 1.02 | 0.760 | 33.58 | −1.51 | 1.536 |
| MV0026-4126-0379 | Mutant | 25.76 | 0.99 | 0.760 | 33.77 | −1.32 | 1.536 |
| MV0026-4126-0280 | Mutant | 25.75 | 0.98 | 0.760 | 33.39 | −1.70 | 1.536 |
| MV0026-4126-0353 | Mutant | 25.73 | 0.96 | 0.760 | 33.79 | −1.30 | 1.536 |
| MV0026-4126-1501 | Mutant | 25.70 | 0.93 | 0.760 | 34.07 | −1.02 | 1.536 |
| MV0026-4126-1466 | Mutant | 25.69 | 0.92 | 0.760 | 33.84 | −1.25 | 1.536 |
| MV0026-4126-1444 | Mutant | 25.67 | 0.90 | 0.760 | 34.60 | −0.49 | 1.536 |
| MV0026-4126-1440 | Mutant | 25.64 | 0.87 | 0.760 | 33.71 | −1.38 | 1.536 |
| MV0026-4126-1412 | Mutant | 25.60 | 0.83 | 0.760 | 34.53 | −0.56 | 1.536 |
| MV0026-4126-1400 | Mutant | 25.51 | 0.74 | 0.760 | 35.18 | 0.09 | 1.536 |
| MV0026-4126-0383 | Mutant | 24.77 | 0.00 | 0.760 | 36.33 | 1.24 | 1.536 |
| MV0026 | Mother | 22.59 | — | — | 39.93 | — | — |
| MV0026-41260001 | Mother | 24.38 | — | — | 36.58 | — | — |
| MV0026-4126-1655 | Mutant | 25.63 | 1.25 | 0.605 | 34.12 | −2.46 | 1.180 |
| MV0026-4126-2643 | Mutant | 25.49 | 1.11 | 0.605 | 34.16 | −2.42 | 1.180 |
| MV0026-4126-1527 | Mutant | 25.48 | 1.10 | 0.605 | 34.48 | −2.10 | 1.180 |
| MV0026-4126-1666 | Mutant | 25.31 | 0.93 | 0.605 | 35.01 | −1.57 | 1.180 |
| MV0026-4126-1657 | Mutant | 25.28 | 0.90 | 0.605 | 34.74 | −1.84 | 1.180 |
| MV0026-4126-1730 | Mutant | 25.28 | 0.90 | 0.605 | 35.05 | −1.53 | 1.180 |
| MV0026-4126-1883 | Mutant | 25.28 | 0.90 | 0.605 | 35.09 | −1.49 | 1.180 |
| MV0026-4126-1559 | Mutant | 25.26 | 0.88 | 0.605 | 34.65 | −1.93 | 1.180 |
| MV0026-4126-1635 | Mutant | 25.12 | 0.74 | 0.605 | 35.63 | −0.95 | 1.180 |
| MV0026-4126-1524 | Mutant | 25.04 | 0.66 | 0.605 | 35.16 | −1.42 | 1.180 |
| MV0026-4126-1740 | Mutant | 25.01 | 0.63 | 0.605 | 34.08 | −2.50 | 1.180 |
| MV0026 | Mother | 22.42 | — | — | 39.95 | — | — |
| MV0026-41260001 | Mother | 23.99 | — | — | 37.08 | — | — |
| MV0026-4126-4613 | Mutant | 26.13 | 2.14 | 0.702 | 33.04 | −4.04 | 1.332 |
| MV0026-4126-4426 | Mutant | 25.47 | 1.48 | 0.702 | 35.11 | −1.97 | 1.332 |
| MV0026-4126-5239 | Mutant | 25.47 | 1.48 | 0.702 | 34.30 | −2.78 | 1.332 |
| MV0026-4126-4510 | Mutant | 25.12 | 1.13 | 0.702 | 34.93 | −2.15 | 1.332 |
| MV0026-4126-4655 | Mutant | 25.06 | 1.07 | 0.702 | 35.19 | −1.89 | 1.332 |
| MV0026-4126-4439 | Mutant | 24.98 | 0.99 | 0.702 | 35.33 | −1.75 | 1.332 |
| MV0026-4126-4475 | Mutant | 24.96 | 0.97 | 0.702 | 34.76 | −2.32 | 1.332 |
| MV0026-4126-4516 | Mutant | 24.91 | 0.92 | 0.702 | 35.14 | −1.94 | 1.332 |
| MV0026-4126-5219 | Mutant | 24.91 | 0.92 | 0.702 | 34.97 | −2.11 | 1.332 |
| MV0026-4126-3297 | Mutant | 24.88 | 0.89 | 0.702 | 35.59 | −1.49 | 1.332 |
| MV0026-4126-4871 | Mutant | 24.81 | 0.82 | 0.702 | 35.30 | −1.78 | 1.332 |
| MV0026-4126-4987 | Mutant | 24.81 | 0.82 | 0.702 | 35.55 | −1.53 | 1.332 |
| MV0026-4126-4562 | Mutant | 24.75 | 0.76 | 0.702 | 36.43 | −0.65 | 1.332 |
| MV0028 | Mother | 19.17 | — | — | 45.43 | | — |
| MV0027-2480 | Mother | 21.37 | — | — | 37.39 | | — |
| MV0027-2480-2118 | Mutant | 23.80 | 2.43 | 0.252 | 32.43 | −4.96 | 0.612 |
| MV0027-2480-2024 | Mutant | 23.37 | 2.00 | 0.252 | 31.43 | −5.96 | 0.612 |
| MV0027-2480-2396 | Mutant | 23.27 | 1.90 | 0.252 | 31.88 | −5.51 | 0.612 |
| MV0027-2480-2293 | Mutant | 23.26 | 1.89 | 0.252 | 32.53 | −4.86 | 0.612 |
| MV0027-2480-2414 | Mutant | 23.24 | 1.87 | 0.252 | 32.59 | −4.80 | 0.612 |
| MV0027-2480-2393 | Mutant | 23.14 | 1.77 | 0.252 | 32.29 | −5.10 | 0.612 |
| MV0027-2480-2620 | Mutant | 23.09 | 1.72 | 0.252 | 32.64 | −4.75 | 0.612 |
| MV0027-2480-2387 | Mutant | 23.08 | 1.71 | 0.252 | 27.39 | −10.00 | 0.612 |
| MV0027-2480-2259 | Mutant | 23.06 | 1.69 | 0.252 | 32.49 | −4.90 | 0.612 |
| MV0027-2480-2693 | Mutant | 23.04 | 1.67 | 0.252 | 32.75 | −4.64 | 0.612 |
| MV0027-2480-2442 | Mutant | 23.03 | 1.66 | 0.252 | 32.54 | −4.85 | 0.612 |
| MV0027-2480-2598 | Mutant | 23.03 | 1.66 | 0.252 | 32.76 | −4.63 | 0.612 |
| MV0027-2480-2319 | Mutant | 22.99 | 1.62 | 0.252 | 32.23 | −5.16 | 0.612 |
| MV0027-2480-2297 | Mutant | 22.97 | 1.60 | 0.252 | 32.95 | −4.44 | 0.612 |
| MV0027-2480-2400 | Mutant | 22.97 | 1.60 | 0.252 | 32.27 | −5.12 | 0.612 |
| MV0027-2480-2541 | Mutant | 22.96 | 1.59 | 0.252 | 32.61 | −4.78 | 0.612 |
| MV0027-2480-2533 | Mutant | 22.93 | 1.56 | 0.252 | 32.03 | −5.36 | 0.612 |
| MV0027-2480-2409 | Mutant | 22.87 | 1.50 | 0.252 | 32.35 | −5.04 | 0.612 |
| MV0027-2480-2550 | Mutant | 22.87 | 1.50 | 0.252 | 32.69 | −4.70 | 0.612 |
| MV0027-2480-2258 | Mutant | 22.86 | 1.49 | 0.252 | 32.58 | −4.81 | 0.612 |
| MV0027-2480-2235 | Mutant | 22.82 | 1.45 | 0.252 | 32.44 | −4.95 | 0.612 |
| MV0027-2480-2373 | Mutant | 22.79 | 1.42 | 0.252 | 32.55 | −4.84 | 0.612 |
| MV0027-2480-2420 | Mutant | 22.75 | 1.38 | 0.252 | 32.34 | −5.05 | 0.612 |
| MV0027-2480-2496 | Mutant | 22.75 | 1.38 | 0.252 | 32.08 | −5.31 | 0.612 |
| MV0027-2480-2367 | Mutant | 22.74 | 1.37 | 0.252 | 31.83 | −5.56 | 0.612 |
| MV0027-2480-2397 | Mutant | 22.64 | 1.27 | 0.252 | 32.63 | −4.76 | 0.612 |
| MV0027-2480-2406 | Mutant | 22.54 | 1.17 | 0.252 | 33.43 | −3.96 | 0.612 |
| MV0027-2480-2283 | Mutant | 22.38 | 1.01 | 0.252 | 32.85 | −4.54 | 0.612 |
| MV0027 | Mother | 19.47 | — | — | 44.91 | | — |
| MV0027-2480 | Mother | 21.82 | — | — | 36.79 | | — |
| MV0027-2480-0781 | Mutant | 23.90 | 2.08 | 0.220 | 32.02 | −4.77 | 0.483 |
| MV0027-2480-0827 | Mutant | 23.28 | 1.46 | 0.220 | 33.05 | −3.74 | 0.483 |
| MV0027-2480-0195 | Mutant | 23.26 | 1.44 | 0.220 | 33.25 | −3.54 | 0.483 |
| MV0027-2480-0978 | Mutant | 23.17 | 1.35 | 0.220 | 33.18 | −3.61 | 0.483 |
| MV0027-2480-0873 | Mutant | 23.08 | 1.26 | 0.220 | 33.54 | −3.25 | 0.483 |
| MV0027-2480-0876 | Mutant | 23.06 | 1.24 | 0.220 | 33.42 | −3.37 | 0.483 |
| MV0027-2480-0836 | Mutant | 23.03 | 1.21 | 0.220 | 33.34 | −3.45 | 0.483 |
| MV0027-2480-0839 | Mutant | 23.03 | 1.21 | 0.220 | 33.99 | −2.80 | 0.483 |
| MV0027-2480-0881 | Mutant | 23.03 | 1.21 | 0.220 | 34.31 | −2.48 | 0.483 |
| MV0027-2480-0919 | Mutant | 23.03 | 1.21 | 0.220 | 33.31 | −3.48 | 0.483 |
| MV0027-2480-0343 | Mutant | 23.01 | 1.19 | 0.220 | 33.64 | −3.15 | 0.483 |
| MV0027-2480-0904 | Mutant | 23.01 | 1.19 | 0.220 | 33.32 | −3.47 | 0.483 |
| MV0027-2480-1027 | Mutant | 23.01 | 1.19 | 0.220 | 33.36 | −3.43 | 0.483 |
| MV0027-2480-0832 | Mutant | 22.96 | 1.14 | 0.220 | 33.40 | −3.39 | 0.483 |
| MV0027-2480-0897 | Mutant | 22.96 | 1.14 | 0.220 | 33.61 | −3.18 | 0.483 |
| MV0027-2480-0823 | Mutant | 22.94 | 1.12 | 0.220 | 33.36 | −3.43 | 0.483 |
| MV0027-2480-1012 | Mutant | 22.93 | 1.11 | 0.220 | 33.22 | −3.57 | 0.483 |
| MV0027-2480-0946 | Mutant | 22.85 | 1.03 | 0.220 | 33.24 | −3.55 | 0.483 |
| MV0027-2480-1026 | Mutant | 22.78 | 0.96 | 0.220 | 33.70 | −3.09 | 0.483 |
| MV0027-2480-0942 | Mutant | 22.75 | 0.93 | 0.220 | 33.96 | −2.83 | 0.483 |
| MV0027-2480-0967 | Mutant | 22.75 | 0.93 | 0.220 | 33.38 | −3.41 | 0.483 |
| MV0027-2480-0879 | Mutant | 22.74 | 0.92 | 0.220 | 33.62 | −3.17 | 0.483 |
| MV0027-2480-0824 | Mutant | 22.73 | 0.91 | 0.220 | 33.81 | −2.98 | 0.483 |
| MV0027-2480-0993 | Mutant | 22.71 | 0.89 | 0.220 | 33.40 | −3.39 | 0.483 |
| MV0027-2480-0867 | Mutant | 22.70 | 0.88 | 0.220 | 33.28 | −3.51 | 0.483 |
| MV0027-2480-0888 | Mutant | 22.64 | 0.82 | 0.220 | 33.88 | −2.91 | 0.483 |
| MV0027-2480-0883 | Mutant | 22.55 | 0.73 | 0.220 | 33.96 | −2.83 | 0.483 |
| MV0027-2480-0954 | Mutant | 22.53 | 0.71 | 0.220 | 34.27 | −2.52 | 0.483 |
| MV0027-2480-0885 | Mutant | 22.09 | 0.27 | 0.220 | 32.78 | −4.01 | 0.483 |
| MV0027 | Mother | 20.06 | — | — | 43.80 | — | — |
| MV0027-2480 | Mother | 21.81 | — | — | 36.97 | — | — |
| MV0027-2480-1093 | Mutant | 23.44 | 1.63 | 0.280 | 32.97 | −4.00 | 0.672 |
| MV0027-2480-1482 | Mutant | 23.40 | 1.59 | 0.280 | 33.43 | −3.54 | 0.672 |
| MV0027-2480-1916 | Mutant | 23.39 | 1.58 | 0.280 | 32.97 | −4.00 | 0.672 |
| MV0027-2480-1058 | Mutant | 23.27 | 1.46 | 0.280 | 33.43 | −3.54 | 0.672 |
| MV0027-2480-1083 | Mutant | 23.27 | 1.46 | 0.280 | 32.98 | −3.99 | 0.672 |
| MV0027-2480-1972 | Mutant | 23.27 | 1.46 | 0.280 | 33.12 | −3.85 | 0.672 |
| MV0027-2480-1530 | Mutant | 23.24 | 1.43 | 0.280 | 33.18 | −3.79 | 0.672 |
| MV0027-2480-1724 | Mutant | 23.24 | 1.43 | 0.280 | 32.93 | −4.04 | 0.672 |
| MV0027-2480-1497 | Mutant | 23.22 | 1.41 | 0.280 | 33.15 | −3.82 | 0.672 |
| MV0027-2480-1697 | Mutant | 23.22 | 1.41 | 0.280 | 33.50 | −3.47 | 0.672 |
| MV0027-2480-1098 | Mutant | 23.20 | 1.39 | 0.280 | 33.38 | −3.59 | 0.672 |
| MV0027-2480-1708 | Mutant | 23.16 | 1.35 | 0.280 | 33.28 | −3.69 | 0.672 |
| MV0027-2480-1521 | Mutant | 23.14 | 1.33 | 0.280 | 32.84 | −4.13 | 0.672 |
| MV0027-2480-1042 | Mutant | 23.12 | 1.31 | 0.280 | 33.24 | −3.73 | 0.672 |
| MV0027-2480-1693 | Mutant | 23.12 | 1.31 | 0.280 | 33.03 | −3.94 | 0.672 |
| MV0027-2480-1659 | Mutant | 23.09 | 1.28 | 0.280 | 33.73 | −3.24 | 0.672 |
| MV0027-2480-1089 | Mutant | 23.08 | 1.27 | 0.280 | 33.23 | −3.74 | 0.672 |
| MV0027-2480-1227 | Mutant | 23.07 | 1.26 | 0.280 | 33.66 | −3.31 | 0.672 |
| MV0027-2480-1785 | Mutant | 23.04 | 1.23 | 0.280 | 33.23 | −3.74 | 0.672 |
| MV0027-2480-1927 | Mutant | 23.02 | 1.21 | 0.280 | 34.16 | −2.81 | 0.672 |
| MV0027-2480-1713 | Mutant | 22.90 | 1.09 | 0.280 | 33.58 | −3.39 | 0.672 |
| MV0027-2480-1409 | Mutant | 22.88 | 1.07 | 0.280 | 34.35 | −2.62 | 0.672 |
| MV0027-2480-1097 | Mutant | 22.87 | 1.06 | 0.280 | 33.46 | −3.51 | 0.672 |
| MV0027-2480-1958 | Mutant | 22.85 | 1.04 | 0.280 | 33.33 | −3.64 | 0.672 |
| MV0027-2480-1040 | Mutant | 22.83 | 1.02 | 0.280 | 33.14 | −3.83 | 0.672 |
| MV0027-2480-1121 | Mutant | 22.81 | 1.00 | 0.280 | 33.84 | −3.13 | 0.672 |
| MV0027-2480-1086 | Mutant | 22.79 | 0.98 | 0.280 | 33.39 | −3.58 | 0.672 |
| MV0027-2480-1105 | Mutant | 22.78 | 0.97 | 0.280 | 33.45 | −3.52 | 0.672 |
| MV0027-2480-1999 | Mutant | 22.67 | 0.86 | 0.280 | 33.68 | −3.29 | 0.672 |
| MV0027-2480-1969 | Mutant | 22.52 | 0.71 | 0.280 | 33.47 | −3.50 | 0.672 |

### Example 3

### Breeding for Elevated Oil in Seed

Crosses between different sources of high oil genes can further increase the oil levels in seed. In addition, subsequent self-pollination of the resulting progeny allows for high oil genes to recombine, potentially producing progeny with increase seed oil levels. The parents for the cross can be elite high oil lines, mutants, or soybeans expressing transgenes to increase seed oil.

### A. Crossing Two High Oil Mutant Breeding Lines

Four populations were developed by inter-crossing previously identified high oil mutants (Tables 1-3). For each population, F₁ seeds were harvested and bulked. F₁ seeds were planted and all F₂ seeds were harvested in bulk. F₂ seeds were planted and a pod was harvested from each F₂ plant and bulked. F₃ seeds were planted and each F₃ plant was harvested individually and evaluated for oil content using NIT. All plants with oil levels greater than one standard deviation from the high seed oil parent were advanced. Each F_{3:4} plot was harvested individually and evaluated for oil content. Lines with oil significantly (p≦0.05) higher than the highest oil parent were selected.

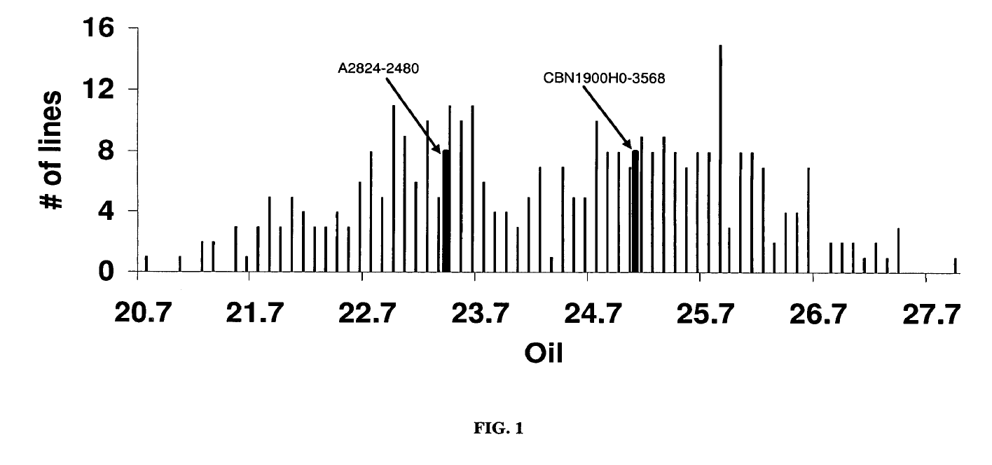

F_{3:4} lines of four populations from crossing two high oil mutants were planted in a two replication test. FIG. 1 shows oil distribution for population MV0026-3568/MV0027-2480.

B. Crossing a High Oil Mutant Breeding Line with an Elite High Oil Variety Expressing Resistance to Glyphosate Herbicide

Two populations were developed by inter-crossing previously identified high oil mutant with elite high oil varieties, MV0028 and MV0029 (Table 1-3). Both MV0028 and MV0029 are transgenic varieties that are resistant to glyphosate herbicide. For each population, F₁ seeds were harvested and bulked. F₁ seeds were planted and all F₂ seeds were harvested in bulk. F₂ seeds were planted and a pod was harvested from each F₂ plant and bulked. F₃ seeds were planted and each F₃ plant was harvested individually and evaluated for oil content using NIT. All plants with oil levels greater than one standard deviation from the high oil parent were advanced. Each F_{3:4} plot was harvested individually and evaluated for oil content. Lines were selected for glyphosate resistance and elevated oil. Oil was considered elevated if oil was significantly (p≦0.05) higher than the highest oil parent.

F_{3:4} lines of four populations from crossing two high oil mutants. Table 9 demonstrates the distribution for populations.

TABLE 9
Protein and oil content of progeny resulting from a mutant
(MV0026) x and high oil elite variety expressing transgenic
herbicide resistance (MV0028 or MV0029) cross.

| Progeny: | Oil | Protein | Yield (% of |
|---|---|---|---|
| (Cross)- No. of Individual | (% DWB) | (% DWB) | check variety) |
| (MV0026/MV0028)-0150 | 24.3 | 37.4 | 80.1 |
| (MV0026/MV0028)-0139 | 24.3 | 38 | 75.3 |
| (MV0026/MV0028)-0027 | 24.3 | 39.5 | 36.1 |
| (MV0026/MV0028)-0299 | 24.2 | 38.3 | 68.2 |
| (MV0026/MV0028)-0300 | 24 | 38.3 | 75.1 |
| (MV0026/MV0028)-0152 | 24 | 38.2 | 110.7 |
| (MV0026/MV0028)-0101 | 24 | 38.5 | 47.1 |
| (MV0026/MV0029)-0016 | 23.9 | 35.9 | 98 |
| (MV0026/MV0028)-0294 | 23.8 | 38.6 | 71.4 |
| (MV0026/MV0028)-0239 | 23.8 | 39.5 | 53.2 |
| (MV0026/MV0029)-0005 | 23.8 | 35.6 | 90.1 |
| (MV0026/MV0028)-0286 | 23.7 | 40.2 | 83.7 |
| (MV0026/MV0028)-0245 | 23.7 | 39.6 | 46.4 |
| (MV0026/MV0028)-0228 | 23.7 | 38.8 | 90.2 |
| (MV0026/MV0028)-0114 | 23.7 | 38.2 | 73.8 |
| (MV0026/MV0029)-0027 | 23.7 | 36.1 | 80.1 |
| (MV0026/MV0028)-0243 | 23.6 | 39.4 | 78 |
| (MV0026/MV0028)-0165 | 23.6 | 39.8 | 63.6 |
| (MV0026/MV0028)-0130 | 23.6 | 39.9 | 75.5 |
| (MV0026/MV0028)-0050 | 23.6 | 38.4 | 89.7 |
| (MV0026/MV0028)-0024 | 23.6 | 39.7 | 72.5 |
| (MV0026/MV0028)-0015 | 23.6 | 39.8 | 72.8 |
| (MV0026/MV0029)-0185 | 23.6 | 39.2 | 74.6 |
| (MV0026/MV0029)-0044 | 23.6 | 40.3 | 74.3 |
| (MV0026/MV0028)-0478 | 23.6 | 38.4 | 42.81 |
| (MV0026/MV0029)-0298 | 23.6 | 37.6 | 108.6 |
| (MV0026/MV0029)-0187 | 23.6 | 36.6 | 78.4 |
| (MV0026/MV0028)-0271 | 23.5 | 39.6 | 66.5 |
| (MV0026/MV0028)-0238 | 23.5 | 39.3 | 98.7 |
| (MV0026/MV0028)-0236 | 23.5 | 39.4 | 55.5 |
| (MV0026/MV0028)-0008 | 23.5 | 38.8 | 77.4 |
| (MV0026/MV0028)-0004 | 23.5 | 40.3 | 36.9 |
| (MV0026/MV0029)-0173 | 23.5 | 37.3 | 85.1 |
| (MV0026/MV0028)-0154 | 23.4 | 37.6 | 61.4 |
| (MV0026/MV0028)-0082 | 23.4 | 39.4 | 54.5 |
| (MV0026/MV0028)-0065 | 23.4 | 40 | 59.6 |
| (MV0026/MV0028)-0063 | 23.4 | 41.1 | 42.6 |
| (MV0026/MV0028)-0047 | 23.4 | 39.8 | 70 |
| (MV0026/MV0028)-0144 | 23.4 | 38.9 | 58.26 |
| (MV0026/MV0028)-0143 | 23.4 | 38.5 | 70.54 |
| (MV0026/MV0029)-0263 | 23.4 | 37.5 | 80.6 |
| (MV0026/MV0029)-0090 | 23.4 | 37.8 | 88.2 |
| (MV0026/MV0028)-0296 | 23.3 | 40.6 | 77.2 |
| (MV0026/MV0028)-0295 | 23.3 | 41.3 | 46.1 |
| (MV0026/MV0028)-0208 | 23.3 | 40.2 | 66.8 |
| (MV0026/MV0028)-0161 | 23.3 | 38.6 | 91.2 |
| (MV0026/MV0028)-0141 | 23.3 | 41.5 | 37.1 |
| (MV0026/MV0028)-0126 | 23.3 | 39.8 | 81.1 |
| (MV0026/MV0028)-0056 | 23.3 | 39.2 | 59.8 |
| (MV0026/MV0029)-0204 | 23.3 | 38.1 | 69.9 |
| (MV0026/MV0029)-0151 | 23.3 | 38.5 | 81.9 |
| (MV0026/MV0029)-0099 | 23.3 | 38.7 | 89.1 |
| (MV0026/MV0029)-0074 | 23.3 | 39.7 | 84 |
| (MV0026/MV0029)-0100 | 23.3 | 38 | 85.6 |
| (MV0026/MV0028)-0248 | 23.2 | 38.6 | 76.7 |
| (MV0026/MV0028)-0179 | 23.2 | 39.8 | 106.3 |
| (MV0026/MV0028)-0118 | 23.2 | 38.8 | 56.7 |
| (MV0026/MV0028)-0068 | 23.2 | 37.9 | 79 |
| (MV0026/MV0028)-0019 | 23.2 | 40.5 | 71.8 |
| (MV0026/MV0028)-0009 | 23.2 | 39 | 79.7 |
| (MV0026/MV0029)-0168 | 23.2 | 39.5 | 57.3 |
| (MV0026/MV0029)-0154 | 23.2 | 38.7 | 94.5 |
| (MV0026/MV0029)-0072 | 23.2 | 38.4 | 82.4 |
| (MV0026/MV0029)-0019 | 23.2 | 38.9 | 92.3 |
| (MV0026/MV0029)-0011 | 23.2 | 38.2 | 97.7 |
| (MV0026/MV0029)-0092 | 23.2 | 40.5 | 84.8 |
| (MV0026/MV0029)-0070 | 23.2 | 39.4 | 72.4 |
| (MV0026/MV0028)-0191 | 23.1 | 39.9 | 86.1 |
| (MV0026/MV0028)-0140 | 23.1 | 40.8 | 36.8 |
| (MV0026/MV0028)-0041 | 23.1 | 39.8 | 53.1 |
| (MV0026/MV0028)-0011 | 23.1 | 38.6 | 92.4 |
| (MV0026/MV0029)-0014 | 23.1 | 40.6 | 77 |
| (MV0026/MV0028)-0499 | 23.1 | 35.8 | 55.43 |
| (MV0026/MV0028)-0032 | 23.1 | 37.5 | 76.89 |
| (MV0026/MV0028)-0005 | 23.1 | 38.7 | 55.26 |
| (MV0026/MV0029)-0213 | 23.1 | 38.8 | 82.7 |
| (MV0026/MV0029)-0182 | 23.1 | 39.1 | 72.9 |
| (MV0026/MV0029)-0116 | 23.1 | 38.8 | 88.2 |
| (MV0026/MV0029)-0106 | 23.1 | 38.9 | 77.9 |
| (MV0026/MV0029)-0071 | 23.1 | 39.2 | 75.3 |
| (MV0026/MV0029)-0015 | 23.1 | 39 | 86.4 |

### Example 4

### Evaluating Soybean for Oil Content

Three techniques (Near Infrared Reflectance (NIR), near-infrared transmittance (NIT), and wet chemistry i.e. solvent extraction) were compared for evaluating the protein and oil composition of soybean seed. Nuclear Magnetic Resonance (NMR) may also be employed. NIR and NIT are non-destructive methods for evaluating composition. Solvent extraction (e.g. Sallee, 1968) is the standard chemical method of for determining oil content of soybean seed. Oil is extracted from finely ground seed with a solvent, such as hexane or petroleum ether, for several hours. All of the extracted substances are considered a part of the oil fraction. The extracted substance can be further analyzed using chromatography.

Five control soybean lines and 10 high oil soybean lines were evaluated for protein and oil using NIR, NIT and solvent extraction. Oil content determined by NIT and NIR were similar to oil content determined by solvent extraction for both control and high oil soybean lines (Table 10).

TABLE 10
Analysis of High Oil (HO) and control soybean seed for Oil, Protein
and Moisture by NIT, NIR and Solvent Extraction Methods.

| | | | | | Moisture | Protein | |
|---|---|---|---|---|---|---|---|
| Soy- | | Oil Content (%) | | | (%) | Content | |
| bean | | | | Solvent | Solvent | (%) | |
| Line | Method | NIT | NIR | Extraction | Extraction | NIT | NIR |
| HO 1 | | 28.70 | 29.19 | 27.00 | 8.35 | 24.81 | 26.61 |
| HO 2 | | 29.50 | 29.96 | 28.30 | 8.24 | 24.61 | 25.99 |
| HO 3 | | 28.30 | 28.30 | 27.70 | 8.33 | 27.73 | 28.68 |
| HO 4 | | 29.30 | 28.93 | 27.40 | 8.23 | 26.62 | 28.88 |
| HO 5 | | 28.70 | 28.35 | 27.40 | 8.14 | 29.02 | 29.15 |
| HO 6 | | 28.20 | 28.02 | 27.40 | 8.20 | 29.30 | 29.85 |
| HO 7 | | 29.60 | 29.31 | 28.30 | 8.25 | 25.60 | 26.30 |
| HO 8 | | 28.70 | 29.04 | 27.60 | 8.24 | 27.99 | 28.71 |
| HO 9 | | 28.90 | 28.77 | 26.70 | 8.28 | 27.06 | 28.59 |
| HO 10 | | 28.70 | 28.72 | 26.90 | 8.25 | 27.49 | 29.01 |
| Control | | 22.40 | 21.55 | 21.10 | 8.46 | 38.76 | 39.10 |
| 1 | | | | | | | |
| Control | | 20.60 | 20.85 | 20.90 | 8.61 | 39.86 | 40.33 |
| 2 | | | | | | | |
| Control | | 19.00 | 18.72 | 18.50 | 8.63 | 45.38 | 45.37 |
| 3 | | | | | | | |
| Control | | 20.80 | 20.91 | 20.40 | 8.54 | 38.06 | 38.99 |
| 4 | | | | | | | |
| Control | | 22.00 | 22.76 | 22.80 | 8.57 | 36.86 | 37.13 |
| 5 | | | | | | | |

* Dry Matter Base

### Example 5

### Processing High Oil Soybeans

Control and high oil soybean were processed to predict the amount of oil that could be extracted from the seed. The seed was processed without use of nitrogen, and extraction was carried out for four hours. After three hours of extraction, the meal was removed from the thimble of the extractor for crushing. After crushing, the meal was returned to the thimble and extraction was carried out for additional one hour. Hexane was distilled off from the miscella. Crude oils generated from both high oil seed and control seed were refined, to produce Refined, Bleached and Deodorized (RBD) oil. Total oil content in high oil seed was measured at around 27.6%. The results from the extraction of the oil, using a soxhlet extractor, yielded 2.6% residual oil in the meal (Table 11). Oil was also extracted from the control and the residual oil in the meal was 0.2%.

TABLE 11
Meal Analysis after Extraction.

| Soybean Line | Control | High Oil |
|---|---|---|
| Moisture | 11.46 | 11.43 |
| Average Oil | 0.2 | 2.6 |
| Average Protein | 52.14 | 37.61 |

In addition, the control and high oil soybean lines were evaluated for fatty acid composition (Table 12). The fatty acid composition of the control and high oil soybean lines were both within the accepted ranges for commercial soy.

TABLE 12
Analysis of Lab Scale Processing of Crude
and RBD Control and High Oil soybean lines.

| | Process | | | |
|---|---|---|---|---|
| | Crude | Deodorized | Crude | Deodorized |
| | Soybean Line | | | |
| | Control | Control | High Oil | High Oil |
| PV, (Meq/Kg) | 1.47 | <0.05 | 1.81 | 0.43 |
| FFA (%) | 0.12 | 0.12 | 0.32 | <0.04 |
| Lovibond AOCS | * | .5R/2.8Y | * | * |
| R/Y Color | | | | |
| p-Anisidine | 0.74 | 0.65 | 0.92 | * |
| value (AV) | | | | |
| Conjugated | 0.16 | 0.38 | 0.14 | * |
| dienes (CD) | | | | |

Fatty Acid (%:)

| | | | | |
|---|---|---|---|---|
| C14:0 | 0.06 | 0.06 | 0.05 | 0.05 |
| C16:0 | 9.85 | 9.74 | 9.5 | 9.53 |
| C16:1n7 | 0.11 | 0.11 | 0.07 | 0.07 |
| C18:0 | 4.09 | 4.06 | 3.97 | 3.97 |
| C18:1 | 21.13 | 21.35 | 18.93 | 19.17 |
| C18:2n6 | 54.97 | 54.9 | 58.38 | 58.22 |
| C18:3n3 | 8.44 | 7.08 | 7.68 | 7.02 |
| C20:0 | 0.31 | 0.31 | 0.32 | 0.31 |
| C20:1n9 | 0.19 | 0.21 | 0.29 | 0.31 |
| C22:0 | 0.34 | 0.32 | 0.38 | 0.35 |
| Others | 0.11 | 0.1 | 0.17 | 0.15 |

Tocopherols (ppm)

| | | | | |
|---|---|---|---|---|
| Alpha | 84.2 | 87.8 | 117.4 | * |
| Gamma/Beta | 812.8 | 757.6 | 1001 | * |
| Delta | 319.7 | 265.8 | 301.1 | * |
| Total | 1216.7 | 1111.2 | 1429.5 | * |

* Not analyzed

The quality of oil extracted from high oil soybeans does not differ from regular soybean. Therefore, processors can obtain greater volumes of quality oil with high oil soybeans compared to typical soybeans, on a per bushel basis. A soybean with 20% oil content produces ˜11.67 lbs of oil/bushel, while a soybean with 28% oil content produces ˜16.41 lbs of oil/bushel. Smaller volumes of high oil soybeans need to be transported to processors to obtain the same amount of oil as typical soybeans, thereby reducing transportation costs associated with producing soybean oil.

Having illustrated and described the principles of the present invention, it should be apparent to persons skilled in the art that the invention can be modified in arrangement and detail without departing from such principles. We claim all modifications that are within the spirit and scope of the appended claims. All publications and published patent documents cited in this specification are incorporated herein by reference to the same extent as if each individual publication or patent application was specifically and individually indicated to be incorporated by reference.

### REFERENCES

The following references, to the extent that they provide exemplary procedural or other details supplementary to those set forth herein, are specifically incorporated herein by reference.
Barwale et al., Planta 167:473-481, 1986.
Cameya et al., Plant Science Letters 21:289-294, 1981.
Cartha et al., Can. J. Bot. 59:1671-1679, 1981.
Cheng et al. Plant Science Letters 19:91-99, 1980.
Fehr, Principles of Cultivar Development Vol. 1, pp. 2-3, 1987.
Honda et al., Euphytica 126:315-320, 2002.
Knothe, J. Am. Oil Chem. Soc. 79: 847-854, 2002.
Liang et al. Acta Botanica Sinica 35:733-738, 1993.
Piper and Boote, J. Am. Oil Chem. Soc. 76:1233-124, 1999.
Ranch et al. In Vitro Cellular & Developmental Biology 21:653-658, 1985.
Saka et al. Plant Science Letters 19:193-201, 1980.
Sallee, Official and Tentative Methods of the American Oil Chemists' Society. Third Ed. American Oil Chemists' Society, Chicago, 1968.
Specht et al., Crop Sci 41:493-509, 2001.
Scott and Kephart, Field Crops Research 49:177-185, 1997.
Tyagi and Hymowitz, Cryo Letters 24: 119-124, 2003.
Widholm et al. In Vitro Selection and Culture-induced Variation in Soybean, In Soybean: Genetics, Molecular Biology and Biotechnology, Eds. Verma and Shoemaker, CAB International, Wallingford, Oxon, England, 1996.
Wright et al., Plant Cell Reports 5: 150-154, 1986.
Yaklich et al., Crop Sci 42:1504-1515, 2002.

1. A soybean plant capable of producing seed with total oil content between 23-35% and wherein said plant and seed comprises one or more transgenic traits.

2. Transgenic soybean seed produced by the plant of claim 1 wherein the total oil content is between 25-33%.

3. Transgenic soybean seed produced by the plant of claim 1 wherein the total oil content is between 27-31%.

4. The soybean plant of claim 1, wherein the transgenic trait confers a preferred property to the soybean plant, comprising one or more phenotypes selected from the group consisting of: herbicide tolerance, increased yield, insect control, fungal disease resistance, virus resistance, nematode resistance, bacterial disease resistance, mycoplasma disease resistance, modified fatty acid composition, increased oil production, modified amino acid composition, modified protein production, increased protein production, increased carbohydrate production, germination and seedling growth control, enhanced animal and human nutrition, low raffinose, drought and/or environmental stress tolerance, altered morphological characteristics, increased digestibility, industrial enzymes, pharmaceutical proteins, peptides and small molecules, improved processing traits, improved flavor, nitrogen fixation, hybrid seed production, reduced allergenicity, biopolymers, biofuels, or any combination of these.

5. Oil extracted from seed of the plant of claim 1.

6. Meal extracted from seed of the plant of claim 1.

7. A method of producing food, feed, fuel or an industrial product comprising the steps of:
(a) obtaining seed from the plant of claim 1;
(b) planting and growing the seed into a mature plant;
(c) harvesting seed from the mature plant; and
(d) preparing food, feed, fuel or an industrial products from the harvested seed.

8. The method of claim 7, wherein the food, feed, fuel or industrial product comprises oil, silage, meal, grain, starch, flour and protein, protein isolate, or soybean hulls.

9. A method of producing a protein product comprising the steps of:
(a) obtaining seed from the plant of claim 1;
(b) planting and growing the seed into a mature plant;
(c) harvesting seed from the mature plant; and
(d) preparing a protein product from the harvested seed.

10. The method of claim 9, wherein the protein product comprises meal, flour, protein isolate, protein concentrate, protein isolate, or soybean hulls.

11. A method of producing an oil product comprising the steps of:
(a) obtaining seed produced from the plant of claim 1;
(b) planting and growing the seed into a mature plant;
(c) harvesting seed from the mature plant; and
(d) preparing food, feed, fuel or an industrial product from the harvested seed.

12. The method of claim 11, wherein the oil product comprises lubricant, food oil, or fuel.

13. A method of producing an industrial product comprising the steps of:
(a) obtaining seed produced from the plant of claim 1;
(b) planting and growing the seed into a mature plant;
(c) harvesting seed from the mature plant; and
(d) fractionating the seed into an industrial product.

14. The method of claim 13, wherein the industrial product comprises fuel, lubricant, resin, binder, glue, adhesive, ink, paint, fungicide, disinfectant, rubber, a cosmetic, a caulking compound, wallboard, anti-foam agent, anti-spattering agent, alcohol, wax, solvent, a dispersing agent, a composite, a plastic, a wetting agent, a cleaner, a protective coating, or a film.

15. The soybean plant of claim 1 capable of producing seeds comprising total oil in excess of 23%, the plant further comprising one or more specialty traits.

16. The plant of claim 15 wherein the specialty trait is selected from the group consisting of: less than 4% linolenic acid, greater than 14% stearic acid, less than 11% palmitic acid, greater than 20% oleic acid, less than 35% linoleic acid, greater than 5% stearidonic acid, greater than 8% alpha-linolenic acid, greater than 8% gamma linolenic acid, greater than 8% docosahexaenoic acid, greater than 8% eicosapentaenoic acid, or greater than 8% docosapentaenoic acid.

17. The plant of claim 15, wherein the specialty trait is obtained by at least one method selected from the group consisting of: mutagenesis, marker-assisted breeding, conventional breeding, or transgenic breeding.

18. A soybean plant capable of producing seed with elevated oil content wherein the total oil content is between 26-35%.

19. The soybean seed of claim 18 wherein the total oil content is between 28-33%.

20. Oil extracted from seed of the plant of claim 18.

21. Meal extracted from seed of said plant of claim 18.

22. A method of producing food or feed comprising the steps of:
(a) obtaining seed from the plant of claim 18;
(b) planting and growing the seed into a mature plant;
(c) harvesting seed from the mature plant; and
(d) preparing a food or feed product from the harvested seed.

23. The method of claim 22, wherein the food or feed product comprises oil, silage, meal, grain, starch, flour and protein, protein isolate, or soybean hulls.

24. A method of producing a protein product comprising the steps of:
(a) obtaining seed from the plant of claim 18;
(b) planting and growing the seed into a mature plant;
(c) harvesting seed from the mature plant; and
(d) preparing a protein product from the harvested seed.

25. The method of claim 24, wherein the protein product comprises meal, flour, protein isolate, protein concentrate, protein isolate or soybean hulls.

26. A method of producing an oil product comprising the steps of:
(a) obtaining seed from the plant of claim 18;
(b) planting and growing the seed into a mature plant;
(c) harvesting seed from the mature plant; and
(d) preparing an oil product from the harvested seed.

27. The method of claim 26, wherein the oil product comprises food oil, lubricant, fuel or an industrial product.

28. A method of producing an industrial product comprising the steps of:
(a) obtaining seed produced from the plant of claim 18;
(b) planting and growing the seed into a mature plant;
(c) harvesting seed from the mature plant; and
(d) preparing an industrial product from the harvested seed.

29. The method of claim 28, wherein the industrial product comprises fuel, lubricant, resin, binder, glue, adhesive, ink, paint, fungicide, disinfectant, rubber, cosmetic, caulking compound, wallboard, an anti-foam agent, an anti-spattering agent, alcohol, wax, solvent, a dispersing agent, a composite, a plastic, a wetting agent, a cleaner, a protective coating, or a film.

30. A method of detecting the presence of a high oil soybean seed in a population of seed, comprising:
(a) obtaining a population of soybean seed; and
(b) detecting in said population the presence of a seed according to claim 2.

31. The method of claim 30, wherein the detection method comprises Near Infrared Reflectance (NIR), Near-Infrared Transmittance (NIT), Nuclear Magnetic Resonance (NMR), or solvent extraction.

32. The soybean plant of claim 18, the plant further comprising one or more specialty traits.

33. The plant of claim 32 wherein the specialty trait is selected from the group consisting of: less than 4% linolenic acid, greater than 14% stearic acid, less than 11% palmitic acid, greater than 20% oleic acid, less than 35% linoleic acid, greater than 5% stearidonic acid, greater than 8% alpha-linolenic acid, greater than 8% gamma linolenic acid, greater than 8% docosahexaenoic acid, greater than 8% eicosapentaenoic acid, or greater than 8% docosapentaenoic acid.

34. The plant of claim 32, wherein the specialty trait is obtained by at least one method selected from the group consisting of: mutagenesis, marker-assisted breeding, conventional breeding, or transgenic breeding.
